(19) United States
(12) Patent Application Publication
(10) Pub. No.: US 2023/0072771 A1
(43) Pub. Date: Mar. 9, 2023

(54) METHODS FOR PRODUCING RETINAL PIGMENT EPITHELIUM CELLS

(71) Applicant: ASTELLAS INSTITUTE FOR REGENERATIVE MEDICINE, Marlborough, MA (US)

(72) Inventors: Yasuhiro Takagi, Marlborough, MA (US); Meng-jiao Shi, Marlborough, MA (US); Mi Sook Chang, Marlborough, MA (US); Irina Klimanskaya, Marlborough, MA (US)

(21) Appl. No.: 17/773,285

(22) PCT Filed: Oct. 28, 2020
(86) PCT No.: PCT/US2020/057654

§ 371 Date: Apr. 29, 2022

Related U.S. Application Data
(60) Provisional application No. 62/928,125, filed on Oct. 30, 2019.

(51) Int. Cl. C12N 5/079; A61P 37/06

(52) U.S. Cl. CPC C12N 5/0621; A61P 37/06; C12N 2501/115; C12N 2501/155; C12N 2533/52; C12N 2533/54; C12N 2501/60

(57) ABSTRACT

The present invention provides an improved method of producing highly pure retinal pigment epithelial (RPE) cells by differentiation of pluripotent stem cells.

### RELATED APPLICATIONS

The instant application is a 35 U.S.C. § 371 national stage filing of International Application No. PCT/US2020/057654, filed on Oct. 28, 2020, which in turn claims priority to U.S. Provisional Application No. 62/928,125, filed on Oct. 30, 2019. The entire contents of which each of the foregoing applications are expressly incorporated herein by reference.

### BACKGROUND

The retinal pigment epithelium (RPE) is the pigmented cell layer just outside the neurosensory retina. This layer of cells nourishes retinal visual cells, and is attached to the underlying choroid (the layer of blood vessels behind the retina) and overlying retinal visual cells. The RPE acts as a filter to determine what nutrients reach the retina from the choroid. Additionally, the RPE provides insulation between the retina and the choroid. Breakdown of the RPE interferes with the metabolism of the retina, causing thinning of the retina. Thinning of the retina can have serious consequences. For example, thinning of the retina may cause “dry” macular degeneration and may also lead to the inappropriate blood vessel formation that can cause “wet” macular degeneration.

Given the importance of the RPE in maintaining visual and retinal health, there have been significant efforts in studying the RPE and in developing methodologies for producing RPE cells in vitro. RPE cells produced in vitro can be used to study the developments of the RPE, to identify factors that cause the RPE to breakdown, or to identify agents that can be used to stimulate repair of endogenous RPE cells. Additionally, RPE cells produced in vitro can themselves be used as a therapy for replacing or restoring all or a portion of a patient's damaged RPE cells. When used in this manner, RPE cells may provide an approach to treat macular degeneration, as well as other diseases and conditions caused, in whole or in part, by damage to the RPE.

In vitro methods for producing retinal pigment epithelial (RPE) cells by inducing differentiation of pluripotent stem cells in the presence of a differentiation-inducing factor in a culture medium are known (see, e.g., Kuroda et al., PLoS One. 2012; 7(5): e37342.). However, these methods require multiple steps combining adhesion culture and floating culture in order to obtain a highly concentrated RPE cell population. These known methods also require a purification step.

Furthermore, using conventionally known methods, when RPE cells are obtained from pluripotent stem cells, cells other than the target cells are generally obtained simultaneously. Consequently, these methods can obtain only a portion of the RPE cells induced in a culture container. Moreover, the purity of the obtained RPE cells is largely influenced by the technique of the experimenter, which makes these methods unsuitable for obtaining a pure population of RPE cells in a short period of time.

Accordingly, there is a need in the art for a simple and efficient method for producing highly pure RPE cells from pluripotent stem cells.

### SUMMARY

The present invention provides an improved method for obtaining retinal pigment epithelial (RPE) from pluripotent stem cells such as human embryonic stem (hES) cells. In particular, the invention is based on the discovery of stages during differentiation of pluripotent stem cells to RPE cells when RPE progenitors can be isolated, partially purified, and further differentiated to mature RPE cells with minimal or without manual picking of the cells. As described herein, following initiation of differentiation of pluripotent cells, the inventors identified time points during the culture process when there is a high percentage of clusters of RPE progenitor cells (e.g., identified as PAX6/MITF positive cells) that stay together. Thus, the methods described herein comprise treatment of the clusters of RPE progenitor cells with a dissociation reagent, such as collagenase or dispase that causes the cells to detach in clusters, followed by size fractionation of the clusters and subsequent subculture of the cells to produce RPE cells. The methods of the invention are both simple and efficient, and result in cultures of RPE cells that are, in some embodiments, substantially pure.

In an aspect, the present invention provides a method for producing a population of retinal epithelium (RPE) cells, the method comprising: (i) obtaining cell clusters of PAX6+/MITF+RPE progenitor cells and dissociating the cell clusters into single cells; (ii) culturing the single cells in a differentiation medium such that the cells differentiate to RPE cells; and (iii) harvesting the RPE cells produced in step (ii); thereby producing a population of RPE cells.

In another aspect, the present invention provides a method for producing a population of retinal epithelium (RPE) cells, the method comprising: (i) obtaining cell clusters of PAX6+/MITF+RPE progenitor cells, (ii) culturing the cell clusters in a differentiation medium such that the cells differentiate to RPE cells; and (iii) harvesting the RPE cells produced in step (ii); thereby producing a population of RPE cells. In any of the embodiments of the present invention, the PAX6+/MITF+RPE progenitor cells may be obtained from a population of pluripotent stem cells.

In an aspect, the present invention provides a method for producing a population of retinal epithelium (RPE) cells, the method comprising: (i) culturing a population of pluripotent stem cells in a first differentiation medium, such that the cells differentiate into RPE progenitor cells; (ii) dissociating the RPE progenitor cells, fractionating the cells to collect RPE progenitor cell clusters, dissociating the RPE progenitor cell clusters into single cells, and subculturing the single cells in a second differentiation medium such that the cells differentiate to RPE cells; and (iii) harvesting the RPE cells produced in step (ii); thereby producing a population of RPE cells. In another aspect, the present invention provides a method for producing a population of retinal epithelium (RPE) cells, the method comprising: (i) culturing a population of pluripotent stem cells in a first differentiation medium, such that the cells differentiate into RPE progenitor cells; (ii) dissociating the RPE progenitor cells, fractionating the cells to collect RPE progenitor cell clusters, and subculturing the collected RPE progenitor cell clusters in a second differentiation medium such that the cells differentiate to RPE cells; and (iii) harvesting the RPE cells produced in step (ii) thereby producing a population of RPE cells. In an embodiment of the present invention, the RPE progenitor cells are positive for PAX6/MITF. In another embodiment, prior to step (i), the pluripotent stem cells are cultured on feeder cells in a medium that supports pluripotency. In a further embodiment, prior to step (i), the pluripotent stem cells are cultured feeder-free in a medium that supports pluripotency. In an embodiment, the medium that supports pluripotency is supplemented with bFGF.

The methods may further comprise harvesting the RPE cells produced in step (ii) in any of the methods described by dissociating the RPE cells, fractionating the RPE cells to collect RPE cell clusters, dissociating the RPE cell clusters into single RPE cells, and culturing the single RPE cells. In another embodiment, the method may further comprise harvesting the RPE cells produced in step (ii) in any of the methods described by dissociating the RPE cells, collecting RPE cell clusters, and selectively picking RPE cell clusters. The method may additionally comprise dissociating the selectively picked RPE cell clusters into single RPE cells and culturing the single RPE cells.

In any of the embodiments of the present invention, the method may further comprise expanding the RPE cells. The RPE cells may be expanded by culturing the cells in maintenance media supplemented with FGF. In an embodiment, the RPE cells are cultured in maintenance medium comprising FGF during the first 1, 2, or 3 days of RPE proliferation at each passage, followed by culturing the RPE cells in maintenance media lacking FGF. In an embodiment, the FGF is added before confluence of RPE cells. In another embodiment, the RPE cells are passaged up to two times.

In any of the embodiments of the present invention, any one of the dissociation steps is carried out by treating the cells with a dissociation reagent. In an embodiment, the dissociation reagent is selected from the group collagenase (such as collagenase I or collagenase IV), accutase, chelator (e.g., EDTA-based dissociation solution), trypsin, dispase, or any combinations thereof.

In any of the embodiments, the pluripotent stem cells are human embryonic stem cells or human induced pluripotent stem cells. In any of the embodiments of the present invention, the population of pluripotent stem cells is embryoid bodies. In any of the embodiments of the present invention, the cells are cultured on feeder cells. In yet another embodiment, the cells are cultured under feeder-free conditions. In a further embodiment, the cells are cultured in a non-adherent culture. In another embodiment, the cells are cultured in an adherent culture.

In an embodiment of the present invention, the differentiation medium is EBDM. In another embodiment, the differentiation medium comprises one or more differentiation agents selected from the group nicotinamide, a transforming factor-β (TGFβ) superfamily (e.g., activin A, activin B, and activin AB), nodal, anti-mullerian hormone (AMH), bone morphogenetic proteins (BMP) (e.g., BMP2, BMP3, BMP4, BMP5, BMP6, and BMP7, growth and differentiation factors (GDF)), WNT pathway inhibitor (e.g., CKI-7, DKK1), a TGF pathway inhibitor (e.g., LDN193189, Noggin), a BMP pathway inhibitor (e.g., SB431542), a sonic hedgehog signal inhibitor, a bFGF inhibitor, and a MEK inhibitor (e.g., PD0325901). In a further embodiment, the differentiation medium comprises nicotinamide. In yet another embodiment, the differentiation medium comprises activin. In an embodiment, the first and second differentiation medium are the same. In another embodiment, the first and second differentiation medium are different. In yet another embodiment, the first and second differentiation medium is EBDM. In an embodiment, the first differentiation medium comprises one or more differentiation agents selected from the group nicotinamide, a transforming factor-β (TGFβ) superfamily (e.g., activin A, activin B, and activin AB), nodal, anti-mullerian hormone (AMH), bone morphogenetic proteins (BMP) (e.g., BMP2, BMP3, BMP4, BMP5, BMP6, and BMP7, growth and differentiation factors (GDF)), WNT pathway inhibitor (e.g., CKI-7, DKK1), a TGF pathway inhibitor (e.g., LDN193189, Noggin), a BMP pathway inhibitor (e.g., SB431542), a sonic hedgehog signal inhibitor, a bFGF inhibitor, and a MEK inhibitor (e.g., PD0325901). In an embodiment, the second differentiation medium comprises one or more differentiation agents selected from the group nicotinamide, a transforming factor-β (TGFβ) superfamily (e.g., activin A, activin B, and activin AB), nodal, anti-mullerian hormone (AMH), bone morphogenetic proteins (BMP) (e.g., BMP2, BMP3, BMP4, BMP5, BMP6, and BMP7, growth and differentiation factors (GDF)), WNT pathway inhibitor (e.g., CKI-7, DKK1), a TGF pathway inhibitor (e.g., LDN193189, Noggin), a BMP pathway inhibitor (e.g., SB431542), a sonic hedgehog signal inhibitor, a bFGF inhibitor, and a MEK inhibitor (e.g., PD0325901). In another embodiment, the first differentiation medium comprises nicotinamide. In another embodiment, the second differentiation medium comprises activin. In any of the embodiments of the present invention, the differentiation medium may further comprise heparin and/or ROCK inhibitor.

In any of the embodiments of the present invention, the cell clusters of RPE progenitor cells are between about 40 μm and about 200 μm in size. In another embodiment, the cell clusters of RPE progenitor cells are between about 40 μm and about 100 μm in size.

In any of the embodiments of the present invention, in step (ii), the cells are cultured on an extracellular matrix selected from the group laminin or a fragment thereof, fibronectin, vitronectin, Matrigel, CellStart, collagen, and gelatin. In an embodiment, the extracellular matrix is laminin or a fragment thereof. In another embodiment, the laminin is selected from laminin-521 and laminin-511. In a further embodiment, the laminin is iMatrix511.

In any of the embodiments of the present invention, the duration of the step of culturing a population of pluripotent stem cells in a first differentiation medium is about 1 week to about 12 weeks. In another embodiment, the duration of the step of culturing a population of pluripotent stem cells in a first differentiation medium is at least about 3 weeks. In another embodiment, the duration of the step of culturing a population of pluripotent stem cells in a first differentiation medium is about 6 to about 10 weeks. In any of the embodiments of the present invention, the duration of culturing in step (ii) is about 1 week to about 8 weeks. In another embodiment, the duration of culturing in step (ii) is at least about 3 weeks. In yet another embodiment, the duration of culturing in step (ii) is about 6 weeks.

In any of the embodiments of the present invention, the RPE progenitor cell clusters or RPE progenitor single cells are subcultured on an extracellular matrix selected from the group laminin, fibronectin, vitronectin, Matrigel, CellStart, collagen, and gelatin. In an embodiment, the extracellular matrix comprises laminin or a fragment thereof. In an embodiment, the laminin or fragment there of is selected from laminin-521 and laminin-511.

In any of the embodiments of the present invention, the single RPE cells are cultured in a medium that supports RPE growth or differentiation. In another embodiment, the single RPE cells are cultured on an extracellular matrix selected from the group laminin or a fragment thereof, fibronectin, vitronectin, Matrigel, CellStart, collagen, and gelatin. In an embodiment, the extracellular matrix is gelatin. In yet another embodiment, the extracellular matrix is laminin or a fragment thereof.

In certain embodiments, the composition of RPE cells comprise a substantially purified population of RPE cells. For example, the composition of RPE cells may contain less than 25%, 20%, 15%, 10%, 9%, 8%, 7%, 6%, 5%, 4%, 3%, 2%, or less than 1% of cells other than RPE cells. In some embodiments, the substantially purified population of RPE cells is one in which the RPE cells comprise at least about 75% of the cells in the population. In other embodiments, a substantially purified population of RPE cells is one in which the RPE cells comprise at least about 80%, 85%, 90%, 91%, 92%, 93%, 94%, 95%, 96%, 97%, 97.5%, 98%, 99%, or even greater than 99% of the cells in the population. In some embodiments, the pigmentation levels of the RPE cells in the cell culture is homogeneous. In other embodiments, the pigmentation of the RPE cells in the cell culture is heterogeneous. A cell culture of the invention may comprise at least about 10¹, 10², 5×10², 10³, 5×10³, 10⁴, 10⁵, 10⁶, 10⁷, 10⁸, 10⁹ or at least about 10¹⁰ RPE cells. In any of the embodiments of the present invention, the RPE cells are human RPE cells.

In any of the embodiments of the present invention, the RPE cell clusters are between about 40 μm and 200 μm in size. In another embodiment, the RPE cell clusters are between about 40 μm and 100 μm in size.

In any of the embodiments of the present invention, the RPE cells express (at the mRNA and/or protein level) one or more (1, 2, 3, 4, 5, 6, 7, 8, 9, 10, or 11) of the following genes: RPE65, CRALBP, PEDF, Bestrophin (BEST1), MITF, OTX2, PAX2, PAX6, premelanosome protein (PMEL or gp-100), tyrosinase, and ZO1. In an embodiment, the RPE cells express Bestrophin, PMEL, CRALBP, MITF, PAX6, and ZO1. In a further embodiment, the RPE cells express Bestrophin, PAX6, MITF, and RPE65. In another embodiment, the RPE cells express MITF and at least one gene selected from Bestrophin and PAX6. In certain embodiments, gene expression is measured by mRNA expression. In other embodiments, gene expression is measured by protein expression.

In any of the embodiments of the present invention, the RPE cells lack substantial expression of one or more stem cell markers. The stem cell markers may be selected from the group OCT4, NANOG, REX1, alkaline phosphatase, SOX2, TDGF-1, DPPA-2, DPPA-4, stage specific embryonic antigen (SSEA)-3 and SSEA-4, tumor rejection antigen (TRA)-1-60 and TRA-1-80. In an embodiment, the RPE cells lack substantial expression of OCT4, SSEA4, TRA-1-81, and alkaline phosphatase. In another embodiment, the RPE cells lack substantial expression of OCT4, NANOG, and SOX2.

In any of the embodiments of the present invention, the RPE cells are cryopreserved following harvesting. In certain embodiments of any of the foregoing aspects, RPE cells are frozen for storage. The cells may be frozen by any appropriate method known in the art, e.g., cryogenically frozen and may be frozen at any temperature appropriate for storage of the cells. In an embodiment, a cryopreserved composition comprises RPE cells and a cryopreservative. Any cryopreservative known in the art may be used, and may comprise one or more of DMSO (dimethyl sulfoxide), ethylene glycol, glycerol, 2-methyl-2-4-pentanediol (MPD), propylene glycol, and sucrose. In an embodiment, the cryopreservative comprises between about 5% to about 50% DMSO and about 30% to about 95% serum, wherein the serum may be optionally fetal bovine serum (FBS). In a particular embodiment, the cryopreservative comprises about 90% FBS and about 10% DMSO. In another embodiment, the cryopreservative comprises about 2% to about 5% DMSO. In an embodiment, the cells may be frozen at approximately −20° C. to −196° C., or at any other temperature appropriate for storage of cells. In an embodiment, the cells are frozen at about −80° C., or at about −196° C. In another embodiment, the cells are frozen at about −135° C. to about −196° C. In a specific embodiment, the cells are frozen at about −135° C. In a further embodiment, the cells may be frozen using an automated slow freezing protocol, whereby the cells are cooled in steps under computer control to a specified temperature. Cryogenically frozen cells are stored in appropriate containers and prepared for storage to reduce risk of cell damage and maximize the likelihood that the cells will survive thawing. In other embodiments, RPE cells are maintained or shipped at about 2° C. to about 37° C. In an embodiment, the RPE cells are maintained or shipped at room temperature, at about 2° C. to about 8° C., at about 4° C., or at about 37° C.

In certain embodiments of any of the foregoing, the method is performed in accordance with current Good Manufacturing Practices (cGMP). In certain embodiments of any of the foregoing, the pluripotent stem cells from which the RPE cells are differentiated were derived in accordance with current Good Manufacturing Practices (cGMP).

The present invention also provides a composition comprising a population of RPE cells produced by the method of any one of the methods described herein. In certain embodiments of any of the foregoing, the method is used to produce a composition comprising at least 10 RPE cells, at least 100 RPE cells, at least 1000 RPE cells, at least 1×10⁴ RPE cells, at least 1×10⁵ RPE cells, at least 5×10⁵ RPE cells, at least 1×10⁶ RPE cells, at least 5×10⁶ RPE cells, at least 1×10⁷ RPE cells, at least 2×10⁷ RPE cells, at least 3×10⁷ RPE cells, at least 4×10⁷ RPE cells, at least 5×10⁷ RPE cells, at least 6×10⁷ RPE cells, at least 7×10⁷ RPE cells, at least 8×10⁷ RPE cells, at least 9×10⁷ RPE cells, at least 1×10⁸ RPE cells, at least 2×10⁸ RPE cells, at least 5×10⁸ RPE cells, at least 7×10⁸ RPE cells, at least 1×10⁹ RPE cells, at least 1×10¹⁰ RPE cells, at least 1×10¹¹ RPE cells, or at least 1×10¹² RPE cells. In an embodiment, the composition comprises about 1×10⁸ to 1×10¹² RPE cells, about 1×10⁹ to 1×10¹¹ RPE cells, or about 5×10⁹ to 1×10¹⁰ RPE cells. In certain embodiments, the number of RPE cells in the composition includes different levels of maturity of RPE cells. In other embodiments, the number of RPE cells in the composition refers to the number of mature RPE cells.

The present invention further provides a method of treating a patient with or at risk of a retinal disease, the method comprising administering an effective amount of a composition comprising a population of RPE cells produced by the method of any one of the methods described herein, or a pharmaceutical composition comprising a population of RPE cells produced by any of the methods described herein and a pharmaceutically acceptable carrier. In an embodiment, the retinal disease is selected from the group retinal degeneration, choroideremia, diabetic retinopathy, age-related macular degeneration (dry or wet), retinal detachment, retinitis pigmentosa, Stargardt's Disease, Angioid streaks, Myopic Macular Degeneration, and glaucoma. In certain embodiments, the method further comprises formulating the RPE cells to produce a composition of RPE cells suitable for transplantation.

In another aspect, the invention provides a method for treating or preventing a condition characterized by retinal degeneration, comprising administering to a subject in need thereof an effective amount of a composition comprising RPE cells, which RPE cells are derived from human embryonic stem cells or other pluripotent stem cells. Conditions characterized by retinal degeneration include, for example, Stargardt's macular dystrophy, age related macular degeneration (dry or wet), diabetic retinopathy, and retinitis pigmentosa. In certain embodiments, the RPE cells are derived from human pluripotent stem cells using one or more of the methods described herein.

In certain embodiments, the preparation is previously cryopreserved and thawed before transplantation.

In certain embodiments, the method of treating further comprises administration of one or more immunosuppressants. In an embodiment, the immunosuppressant may comprise one or more of: anti-lymphocyte globulin (ALG) polyclonal antibody, anti-thymocyte globulin (ATG) polyclonal antibody, azathioprine, BASILIXIMAB® (anti-IL-2Ra receptor antibody), cyclosporin (cyclosporin A), DACLIZUMAB® (anti-IL-2Ra receptor antibody), everolimus, mycophenolic acid, RITUX1MAB® (anti-CD20 antibody), sirolimus, tacrolimus, and mycophemolate mofetil (MMF). When immunosuppressants are used, they may be administered systemically or locally, and they may be administered prior to, concomitantly with, or following administration of the RPE cells. In certain embodiments, immunosuppressive therapy continues for weeks, months, years, or indefinitely following administration of RPE cells. In other embodiments, the method of treatment does not require administration of immunosuppressants. In certain embodiments, the method of treatment comprises administration of a single dose of RPE cells. In other embodiments, the method of treatment comprises a course of therapy where RPE cells are administered multiple times over some period. Exemplary courses of treatment may comprise weekly, biweekly, monthly, quarterly, biannually, or yearly treatments. Alternatively, treatment may proceed in phases whereby multiple doses are required initially (e.g., daily doses for the first week), and subsequently fewer and less frequent doses are needed. Numerous treatment regimens are contemplated.

In certain embodiments, a composition comprising RPE cells is transplanted in a suspension, matrix or substrate. In certain embodiments, the composition is administered by injection into the subretinal space of the eye. In certain embodiments, about 10⁴ to about 10⁶ RPE cells are administered to the subject. In certain embodiments, the method further comprises the step of monitoring the efficacy of treatment or prevention by measuring electroretinogram responses, optomotor acuity threshold, or luminance threshold in the subject. The method may also comprise monitoring the efficacy of treatment or prevention by monitoring immunogenicity of the cells or migration of the cells in the eye. In other embodiments, the effectiveness of treatment may be assessed by determining the visual outcome by one or more of: slit lamp biomicroscopic photography, fundus photography, 1VFA, and SD-OCT, and best corrected visual acuity (BCVA). The method may produce an improvement in corrected visual acuity (BCVA) and/or an increase in letters readable on a visual acuity chart, such as the Early Treatment Diabetic Retinopathy Study (ETDRS).

In certain aspects, the invention provides a pharmaceutical composition for treating or preventing a condition characterized by retinal degeneration, comprising an effective amount of RPE cells, which RPE cells are derived from human embryonic stem cells or other pluripotent stem cells. The pharmaceutical composition may be formulated in a pharmaceutically acceptable carrier according to the route of administration. For example, the preparation may be formulated for administration to the subretinal space of the eye. The composition may comprise at least 10³, 10⁴, 10⁵, 5×10⁵, 6×10⁵, 7×10⁵, 8×10⁵, 9×10⁵, 10⁶, 2×10⁶, 3×10⁶, 4×10⁶, 5×10⁶, 6×10⁶, 7×10⁶, 8×10⁶, 9×10⁶, or 10⁷ RPE cells. In certain embodiments, the composition may comprise at least 1×10⁴, 5×10⁴, 1×10⁵, 1.5×10⁵, 2×10⁵, 3×10⁵, 4×10⁵, 5×10⁵, 6×10⁵, 7×10⁵, 8×10⁵, 9×10⁵, 1×10⁶ RPE cells.

In certain embodiments, the RPE cells are formulated in a pharmaceutical composition comprising RPE cells and a pharmaceutically acceptable carrier or excipient. In certain embodiments, the invention provides a pharmaceutical preparation comprising human RPE cells derived from human embryonic stem cells or other pluripotent stem cells. Pharmaceutical preparations may comprise at least about 10¹, 10², 5×10², 10³, 5×10³, 10⁴, 5×10⁴, 10⁵, 1.5×10⁵, 2×10⁵, 5×10⁵, 10⁶, 10⁷, 10⁸, 10⁹ or about 10¹⁰ hRPE cells.

In another aspect, the invention provides a method for screening to identify agents that modulate the survival of RPE cells. For example, RPE cells obtained from human embryonic stem cells can be used to screen for agents that promote RPE survival. Identified agents can be used, alone or in combination with RPE cells, as part of a treatment regimen. Alternatively, identified agents can be used as part of a culture method to improve the survival of RPE cells differentiated in vitro.

In another aspect, the invention provides a method for screening to identify agents that modulate RPE cell maturity. For example, RPE cells obtained from human ES cells can be used to screen for agents that promote RPE maturation.

### BRIEF DESCRIPTION OF DRAWINGS

FIG. 1 shows a time course of PAX6 and MITF mRNA expression by qPCR in RPE progenitor cells relative to normalized GAPDH mRNA expression.

FIG. 2 shows a time course of PAX6 and MITF expression by immunofluorescence assay (IFA) of various cell fractions obtained after initiation of differentiation to RPE cells.

FIG. 3 shows schematic diagrams of the single RPE progenitor cell subculture method (FIG. 3A) and the RPE progenitor cell cluster subculture method (FIG. 3B).

FIG. 4 shows an exemplary workflow of the single RPE progenitor cell subculture method and the RPE progenitor cell cluster subculture method.

FIG. 5 shows the characteristics of RPE cells obtained by the single RPE progenitor cell subculture and RPE progenitor cell cluster subculture methods in accordance with embodiments of the invention.

### DETAILED DESCRIPTION

The present invention provides improved methods for obtaining retinal pigment epithelial (RPE) cells from pluripotent stem cells such as human embryonic stem (hES) cells, embryo-derived cells, and induced pluripotent stem cells (iPS cells). In particular, the invention is based on the discovery of stages during differentiation of pluripotent stem cells when RPE progenitors can be isolated, partially purified, and further differentiated to mature RPE cells with minimal, selective picking or without manual picking of the cells. In particular, as described herein, following initiation of differentiation of pluripotent cells, the inventors identified time points during the culture process when there is sufficient number of clusters of RPE progenitor cells (identified as PAX6/MITF positive cells) that stay together when the culture is dissociated with a dissociation reagent, such as collagenase and dispase. The cultures are not over-mature, so that most of the non-RPE cells in culture or adhered to such RPE progenitor cell clusters can be eliminated as single cells. Additionally, large clusters of non-RPE cells as well as clusters containing a mixture of RPEs and non-RPEs may be eliminated by size fractionation, allowing for increased purity. Thus, the methods described herein comprise treatment of the clusters of RPE progenitor cells with a dissociation reagent, such as collagenase or dispase, followed by size fractionation to isolate RPE progenitor cell clusters of a particular size, and subculture of the RPE progenitor cells as single cells or as cell clusters to produce RPE cells.

In an embodiment, the methods of the invention comprise isolating RPE progenitor cell clusters which are between about 40 to about 200 μm, or between about 40 and about 100 μm in size. In an embodiment, the RPE progenitor cell clusters are collected by using a cell strainer or a series of cell strainers and collecting the cell clusters having the desired size requirement. For example, to obtain a cell cluster between about 40 to about 200 μm or between about 40 to about 100 μm, cell strainers of 40 μm, 70 μmm, 100 μm, 200 μm or any other filter size that would allow obtaining the desired cell cluster size may be used. The methods of the invention are both simple and efficient. In some embodiments, the methods of the invention result in cultures of RPE cells that are substantially pure. A substantially purified population of RPE cells is one in which the RPE cells comprise at least about 75% of the cells in the population. In other embodiments, a substantially purified population of RPE cells is one in which the RPE cells comprise at least about 80%, 85%, 90%, 91%, 92%, 93%, 94%, 95%, 96%, 97%, 97.5%, 98%, 98.5, 99%, or even greater than 99% of the cells in the population.

The current invention provides several advantages over methods known in the art for producing RPE cells, including, for example, greatly enhanced RPE cell yields, greatly enhanced RPE cell purity, improved ease of manual RPE cell isolation, the ability for automated RPE cell selection, the absence of the requirement for any further purification by manual or automated selection, and the use of simple constituents, which enables commercial large-scale manufacturing. In some embodiments, the methods of the invention increase the yield of RPE, e.g., up to more than 50-90 times greater, as compared to cells produced by the conventional manufacturing method involving manual picking, and produces RPE cells with high consistency of purity over 98% to 99%.

In order to make the invention described herein fully understood, the following detailed description are provided. Various embodiments of the invention have been described in detail, and may be further illustrated by the examples provided herein. All technical and scientific terms used herein unless otherwise defined, have the same meaning as those skilled in the art to which the invention pertains generally understood.

### Definitions

Unless otherwise specified, each of the following terms have the meaning set forth in this section.

The indefinite articles “a” and “an” refer to at least one of the associated noun, and are used interchangeably with the terms “at least one” and “one or more.”

The conjunctions “or” and “and/or” are used interchangeably as non-exclusive disjunctions.

As used herein, the term “retinal pigment epithelial cell” or “RPE cell” are used interchangeably herein to refer to an epithelial cell constituting the retinal pigment epithelium. The term is used generically to refer to differentiated RPE cells, regardless of the level of maturity of the cells, and thus may encompass RPE cells of various levels of maturity. RPE cells can be visually recognized by their cobblestone morphology and the initial appearance of pigment. RPE cells can also be identified molecularly based on substantial lack of expression of embryonic stem cell markers such as OCT4 and NANOG, as well as based on the expression of RPE markers such as RPE65, PEDF, CRALBP, and/or bestrophin (BEST1). In one embodiment, the RPE cells lack substantial expression of one or more of embryonic stem cell markers including but not limited to OCT4, NANOG, REX1, alkaline phosphatase, SOX2, TDGF-1, DPPA-2, DPPA-4, stage specific embryonic antigen (SSEA)-3 and SSEA-4, tumor rejection antigen (TRA)-1-60 and/or TRA-1-80. In another embodiment, the RPE cells express one or more RPE cell markers including but not limited to RPE65, CRALBP, PEDF, Bestrophin, MITF, OTX2, PAX2, PAX6, premelanosome protein (PMEL or gp-100), and/or tyrosinase. In another embodiment, the RPE cells express ZO1. In an embodiment, the RPE cells express MITF and at least one marker selected from Bestrophin and PAX6. Note that when other RPE-like cells are referred to, they are generally referred to as adult RPEs, fetal RPEs, primary cultures of adult or fetal RPEs, and immortalized RPE cell lines such as APRE19 cells. Thus, unless otherwise specified, RPE cells, as used herein, refers to RPE cells obtained from pluripotent stem cells (PSC-RPE) and may refer to RPE cells obtained from human pluripotent stem cells (hRPE).

Pigmentation of the RPE cells may vary with cell density in the culture and the maturity of the RPE cells. However, when cells are referred to as pigmented, the term is understood to refer to any and all levels of pigmentation. Thus, the present invention provides RPE cells with varying degrees of pigmentation. In certain embodiments, the pigmentation of a RPE is the same as the average pigmentation as other RPE-like cells, such as adult RPEs, fetal RPEs, primary cultures of adult or fetal RPEs, or immortalized RPE cell lines such as ARPE19. In certain embodiments, the degree of pigmentation of a RPE is higher than the average pigmentation of other RPE-like cells, such as adult RPEs, fetal RPEs, primary cultures of adult or fetal RPEs, or immortalized RPE cell lines such as ARPE19. In certain other embodiments, the degree of pigmentation of a RPE is lower than of the average pigmentation of other RPE-like cells, such as adult RPEs, fetal RPEs, primary cultures of adult or fetal RPEs, or immortalized RPE cell lines such as ARPE19.

Functional evaluation of RPE cells can be confirmed using, for example, secretability, phagocytic capacity and the like of a cytokine (VEGF or PEDF, etc.), phagocytosis of shed rod and cone outer segments (or phagocytosis of another substrate, such as polystyrene beads), absorption of stray light, vitamin A metabolism, regeneration of retinoids, trans-epithelial resistance, cell polarity, and tissue repair. Evaluation may also be performed by testing in vivo function after RPE cell implantation into a suitable host animal (such as a human or non-human animal suffering from a naturally occurring or induced condition of retinal degeneration), e.g., using behavioral tests, fluorescent angiography, histology, tight junctions conductivity, or evaluation using electron microscopy. These functional evaluation and confirmation operations can be performed by those of ordinary skill in the art. RPE cells, as used herein, include human RPE (hRPE) cells.

As used herein, the term “progenitor cell of an RPE cell” or “RPE progenitor cell” are used interchangeably herein to refer to a cell directed to differentiate into a retinal cell. In an embodiment, the term RPE progenitor cell may be used to refer to any cell directed to differentiate into a retinal cell up to harvesting the RPE cell (e.g., for plating at P0 as described herein). It will be appreciated that in the latter stages of differentiation, the differentiation culture may comprise a mixture of RPE progenitor cells and RPE cells. In an embodiment, a progenitor cell expresses (MITF (pigment epithelial cell, progenitor cell), PAX6 (progenitor cell), Rx (retinal progenitor cell), Crx (photoreceptor precursor cell), and/or Chx10 (bipolar cell) etc.) and the like. In an embodiment, the RPE progenitor cell expresses PAX6 and MITF.

The terms “mature RPE cell” and “mature differentiated RPE cell” are used interchangeably throughout to refer to changes that occur following initial differentiation of RPE cells. Specifically, although RPE cells may be recognized, in part, based on initial appearance of pigment, after differentiation mature RPE cells may be recognized based on enhanced pigmentation. Pigmentation post-differentiation may not be indicative of a change in the RPE state of the cells (e.g., the cells are still differentiated RPE cells). The changes in pigment post-differentiation may correspond to the density at which the RPE cells are cultured and maintained. Mature RPE cells may have increased pigmentation that accumulates after initial differentiation. Mature RPE cells may be more pigmented than immature RPE cells and may appear after the RPEs stop proliferating, for example, due to high cell density within the culture dish. Mature RPE cells may be subcultured at a lower density such that it allows proliferation of the mature RPE cells. Proliferation of the mature RPEs in culture may be accompanied by dedifferentiation—loss of pigment and epithelial morphology, both of which are restored after the cells form a monolayer and become quiescent. In this context, mature RPE cells may be cultured to produce RPE cells. Such RPE cells are still differentiated RPE cells that express markers of RPE. Thus, in contrast to the initial appearance of pigmentation that occurs when RPE cells begin to appear, pigmentation changes post-differentiation are phenomenological and do not reflect dedifferentiation of the cells away from an RPE fate. Changes in pigmentation post-differentiation may also correlate with changes in one or more of PAX2, PAX6, tyrosinase, neural markers such as tubulin beta III, bestrophin, RPE65, and CRALBP expression. In an embodiment, changes in pigmentation post-differentiation shows a reverse correlation with one or more of PAX6 and neural markers (such as tubulin beta III). In another embodiment, changes in pigmentation post-differentiation shows a direct correlation with RPE65 and CRALBP.

As used herein, the term “pluripotent stem cells”, “PS cells”, or “PSCs” includes embryonic stem cells, induced pluripotent stem cells, and embryo-derived pluripotent stem cells, regardless of the method by which the pluripotent stem cells are derived. Pluripotent stem cells are defined functionally as stem cells that: (a) are capable of inducing teratomas when transplanted in immunodeficient (SCID) mice; (b) are capable of differentiating to cell types of all three germ layers (e.g., can differentiate to ectodermal, mesodermal, and endodermal cell types); (c) express one or more markers of embryonic stem cells (e.g., express OCT4, alkaline phosphatase, SSEA-3 surface antigen, SSEA-4 surface antigen, NANOG, TRA-1-60, TRA-1-81, SOX2, REX1, etc); and d) are capable of self-renewal. The term “pluripotent” refers to the ability of a cell to form all lineages of the body or soma (i.e., the embryo proper). For example, embryonic stem cells and induced pluripotent stem cells are a type of pluripotent stem cells that are able to form cells from each of the three germs layers: the ectoderm, the mesoderm, and the endoderm. Pluripotency is a continuum of developmental potencies ranging from the incompletely or partially pluripotent cell which is unable to give rise to a complete organism to the more primitive, more pluripotent cell, which is able to give rise to a complete organism (e.g., an embryonic stem cell). Exemplary pluripotent stem cells can be generated using, for example, methods known in the art. Exemplary pluripotent stem cells include, but are not limited to, embryonic stem cells derived from the ICM of blastocyst stage embryos, embryonic stem cells derived from one or more blastomeres of a cleavage stage or morula stage embryo (optionally without destroying the remainder of the embryo), induced pluripotent stem cells produced by reprogramming of somatic cells into a pluripotent state, and pluripotent cells produced from embryonic germ (EG) cells (e.g., by culturing in the presence of FGF-2, LIF and SCF). Such embryonic stem cells can be generated from embryonic material produced by fertilization or by asexual means, including somatic cell nuclear transfer (SCNT), parthenogenesis, and androgenesis.

In an embodiment, pluripotent stem cells may be genetically engineered or otherwise modified, for example, to increase longevity, potency, homing, to prevent or reduce immune responses, or to deliver a desired factor in cells that are obtained from such pluripotent cells (for example, RPEs). For example, the pluripotent stem cell and therefore, the resulting differentiated cell, can be engineered or otherwise modified to lack or have reduced expression of beta 2 microglobulin, HLA-A, HLA-B, HLA-C, TAP1, TAP2, Tapasin, CTIIA, RFX5, TRAC, or TRAB genes. The pluripotent stem cell and the resulting differentiated cell may be engineered or otherwise modified to increase expression of a gene. There are a variety of techniques for engineering cells to modulate the expression of one or more genes (or proteins), including the use of viral vectors such as AAV vectors, zinc-finger nucleases (ZFNs), transcription activator-like effector nucleases (TALENs), and CRISPR/Cas-based methods for genome engineering, as well as the use of transcriptional and translational inhibitors such as antisense and RNA interference (which can be achieved using stably integrated vectors and episomal vectors).

The term “embryo” or “embryonic” is meant a developing cell mass that has not been implanted into the uterine membrane of a maternal host. An “embryonic cell” is a cell isolated from or contained in an embryo. This also includes blastomeres, obtained as early as the two-cell stage, or aggregated blastomeres after extraction.

The term “embryo-derived cells” (EDC), as used herein, refers broadly to morula-derived cells, blastocyst-derived cells including those of the inner cell mass, embryonic shield, or epiblast, or other pluripotent stem cells of the early embryo, including primitive endoderm, ectoderm, and mesoderm and their derivatives. “EDC” also including blastomeres and cell masses from aggregated single blastomeres or embryos from varying stages of development, but excludes human embryonic stem cells that have been passaged as cell lines.

The term “embryonic stem cells”, “ES cells,” or “ESCs” as used herein, refer broadly to cells isolated from the inner cell mass of blastocysts or morulae and that have been serially passaged as cell lines. The term also includes cells isolated from one or more blastomeres of an embryo, preferably without destroying the remainder of the embryo (see, e.g., Chung et al., Cell Stem Cell. 2008 Feb. 7; 2(2): 1 13-7; U.S. Pub No. 20060206953; U.S. Pub No. 2008/0057041, each of which is hereby incorporated by reference in its entirety). The ES cells may be derived from fertilization of an egg cell with sperm or DNA, nuclear transfer, parthenogenesis, or by any means to generate ES cells with homozygosity in the HLA region. ES cells may also refer to cells derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce a cell. In an embodiment, the embryonic stem cell may be a human embryonic stem cell (or “hES cells”). In an embodiment, human embryonic stem cells are not derived from embryos over 14 days from fertilization. In another embodiment, human embryonic stem cells are not derived from embryos that have been developed in vivo. In another embodiment, human embryonic stem cells are derived from preimplantation embryos produced by in vitro fertilization.

“Induced pluripotent stem cells” or “iPS cells,” as used herein, generally refer to pluripotent stem cells obtained by reprogramming a somatic cell. An iPS cell may be generated by expressing or inducing expression of a combination of factors (“reprogramming factors”), for example, OCT4 (sometimes referred to as OCT 3/4), SOX2, MYC (e.g., c-MYC or any MYC variant), NANOG, LIN28, and KLF4, in a somatic cell. In an embodiment, the reprogramming factors comprise OCT4, SOX2, c-MYC, and KLF4. In another embodiment, reprogramming factors comprise OCT4, SOX2, NANOG, and LIN28. In certain embodiments, at least two reprogramming factors are expressed in a somatic cell to successfully reprogram the somatic cell. In other embodiments, at least three reprogramming factors are expressed in a somatic cell to successfully reprogram the somatic cell. In other embodiments, at least four reprogramming factors are expressed in a somatic cell to successfully reprogram the somatic cell. In another embodiment, at least five reprogramming factors are expressed in a somatic cell to successfully reprogram the somatic cell. In yet another embodiment, at least six reprogramming factors are expressed in the somatic cell, for example, OCT4, SOX2, c-MYC, NANOG, LIN28, and KLF4. In other embodiments, additional reprogramming factors are identified and used alone or in combination with one or more known reprogramming factors to reprogram a somatic cell to a pluripotent stem cell.

iPS cells may be generated using fetal, postnatal, newborn, juvenile, or adult somatic cells. Somatic cells may include, but are not limited to, fibroblasts, keratinocytes, adipocytes, muscle cells, organ and tissue cells, and various blood cells including, but not limited to, hematopoietic cells (e.g., hematopoietic stem cells). In an embodiment, the somatic cells are fibroblast cells, such as a dermal fibroblast, synovial fibroblast, or lung fibroblast, or a non-fibroblastic somatic cell.

iPS cells may be obtained from a cell bank. Alternatively, iPS cells may be newly generated by methods known in the art. iPS cells may be specifically generated using material from a particular patient or matched donor with the goal of generating tissue-matched cells. In an embodiment, iPS cells may be universal donor cells that are not substantially immunogenic.

The induced pluripotent stem cell may be produced by expressing or inducing the expression of one or more reprogramming factors in a somatic cell. Reprogramming factors may be expressed in the somatic cell by infection using a viral vector, such as a retroviral vector or other gene editing technologies, such as CRISPR, Talen, zinc-finger nucleases (ZFNs). Also, reprogramming factors may be expressed in the somatic cell using a non-integrative vector, such as an episomal plasmid, or RNA, such as synthetic mRNA or via an RNA virus such as Sendai virus. When reprogramming factors are expressed using non-integrative vectors, the factors may be expressed in the cells using electroporation, transfection, or transformation of the somatic cells with the vectors. For example, in mouse cells, expression of four factors (OCT3/4, SOX2, c-MYC, and KLF4) using integrative viral vectors is sufficient to reprogram a somatic cell. In human cells, expression of four factors (OCT3/4, SOX2, NANOG, and LIN28) using integrative viral vectors is sufficient to reprogram a somatic cell.

Expression of the reprogramming factors may be induced by contacting the somatic cells with at least one agent, such as a small organic molecule agents, that induce expression of reprogramming factors.

The somatic cell may also be reprogrammed using a combinatorial approach wherein the reprogramming factor is expressed (e.g., using a viral vector, plasmid, and the like) and the expression of the reprogramming factor is induced (e.g., using a small organic molecule).

Once the reprogramming factors are expressed or induced in the cells, the cells may be cultured. Over time, cells with ES characteristics appear in the culture dish. The cells may be chosen and subcultured based on, for example, ES cell morphology, or based on expression of a selectable or detectable marker. The cells may be cultured to produce a culture of cells that resemble ES cells.

To confirm the pluripotency of the iPS cells, the cells may be tested in one or more assays of pluripotency. For examples, the cells may be tested for expression of ES cell markers; the cells may be evaluated for ability to produce teratomas when transplanted into SCID mice; the cells may be evaluated for ability to differentiate to produce cell types of all three germ layers.

iPS cells may be from any species. These iPS cells have been successfully generated using mouse and human cells. Furthermore, iPS cells have been successfully generated using embryonic, fetal, newborn, and adult tissue. Accordingly, one may readily generate iPS cells using a donor cell from any species. Thus, one may generate iPS cells from any species, including but not limited to, human, non-human primates, rodents (mice, rats), ungulates (cows, sheep, etc.), dogs (domestic and wild dogs), cats (domestic and wild cats such as lions, tigers, cheetahs), rabbits, hamsters, goats, elephants, panda (including giant panda), pigs, raccoon, horse, zebra, marine mammals (dolphin, whales, etc.) and the like.

As used herein, the term “differentiation” is the process by which an unspecialized (“uncommitted”) or less specialized cell acquires the features of a specialized cell such as, for example, an RPE cell. A differentiated cell is one that has taken on a more specialized position within the lineage of a cell. For example, an hES cell can be differentiated into various more differentiated cell types, including an RPE cell.

As used herein, the term “cultured” or “culturing” refers to the placing of cells in a medium containing, among other things nutrients needed to sustain the life of the cultured cells, any specified added substances. Cells are cultured “in the presence of” a specified substance when the medium in which such cells are maintained contains such specified substance. Culturing can take place in any vessel or apparatus in which the cells can be maintained exposed to the medium, including without limitation petri dishes, culture dishes, blood collection bags, roller bottles, flasks, test tubes, microtiter wells, hollow fiber cartridges or any other apparatus known in the art.

As used herein, the term “subculturing” or “passaging,” refers to transferring some or all cells from a previous culture to fresh growth medium and/or plating onto a new culture dish and further culturing the cells. Subculturing may be done, e.g., to prolong the life, enrich for a desired cell population, and/or expand the number of cells in the culture. For example, the term includes transferring, culturing, or plating some or all cells to a new culture vessel at a lower cell density to allow proliferation of the cells.

As used herein, the term “selectively picking” or “selective picking” refers to mechanically picking or separating a subset of cells from a larger population based on visual or other phenotypic characteristic. Selective picking may be performed manually or by an automated system, and may be performed with the aid of a microscope, computer imaging system, or other means for identifying the cells to be picked.

As used herein, the term “dissociation reagent” refers to an enzymatic or non-enzymatic reagent that promotes cell dissociation or detachment into cell aggregates or into single cells. Examples of dissociation reagents include, but are not limited to, collagenase (such as collagenase I or collagenase IV), accutase, chelator (e.g., EDTA-based dissociation solution), trypsin, dispase, or any combinations thereof.

As used herein, the term “extracellular matrix” refers to any substance to which cells can adhere in culture and typically contains extracellular components to which the cells can attach and thus it provides a suitable culture substrate. Suitable for use with the present invention are extracellular matrix components derived from basement membrane or extracellular matrix components that form part of adhesion molecule receptor-ligand couplings. Examples of an extracellular matrix includes, but is not limited to, laminin or a fragment thereof, e.g., laminin 521, laminin 511, or iMatrix511, fibronectin, vitronectin, Matrigel, CellStart, collagen, gelatin, proteoglycan, entactin, heparin sulfate, and the like, alone or in various combinations.

As used herein, the term “laminin” refers to a heterotrimer molecule consisting of α, β, γ chains, or fragments thereof, which is an extracellular matrix protein containing isoforms having different subunit chain compositions. Specifically, laminin has about 15 kinds of isoforms including heterotrimers of combinations of 5 kinds of a chain, 4 kinds of β chain and 3 kinds of γ chain. The number of each of α chain (α1-α5), β chain (β1-β4) and γ chain (γ1-γ3) is combined to determine the name of a laminin. For example, a laminin composed of a combination of α1 chain, β1 chain, γ1 chain is named laminin-111, a laminin composed of a combination of α5 chain, β1 chain, γ1 chain is named laminin-511, and a laminin composed of a combination of α5 chain, β2 chain, γ1 chain is named laminin-521. A laminin derived from a mammal can be used in the methods of the invention. Examples of mammals include mouse, rat, guinea pig, hamster, rabbit, cat, dog, sheep, swine, bovine, horse, goat, monkey and human. Human laminin is preferably used when RPE cells are produced. In an embodiment, the laminin is a recombinant laminin. Currently, human laminin is known to include 15 kinds of isoforms.

Any laminin fragment may be used in the present invention as long as it retains the function of each corresponding laminin. That is, a “laminin fragment” used in the present invention is not limited as to the length of each chain as long as it is a molecule having laminin α chain, β chain and γ chain constituting a heterotrimer, retaining binding activity to integrin, and maintaining cell adhesion activity. A laminin fragment shows integrin binding specificity that varies for the original laminin isoform, and can exert an adhesion activity to a cell that expresses the corresponding integrin. In an embodiment, the laminin is a recombinant laminin-511 E8 fragment (e.g., iMatrix-511 (Takara Bio)).

As used herein, “administration”, “administering” and variants thereof refers to introducing a composition or agent into a subject and includes concurrent and sequential introduction of a composition or agent. “Administration” can refer, e.g., to therapeutic, pharmacokinetic, diagnostic, research, placebo, and experimental methods. “Administration” also encompasses in vitro and ex vivo treatments. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the terms “subject”, “individual”, “host”, and “patient” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in particular embodiments the subject is a human.

As used herein, the terms “therapeutic amount”, “therapeutically effective amount”, an “amount effective”, or “pharmaceutically effective amount” of an active agent (e.g., an RPE cell) are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, anticipated cell engraftment, long term survival, and/or the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For the therapeutic agents described herein (e.g., RPE cells), a therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

Pharmacokinetic principles provide a basis for modifying a dosage regimen to obtain a desired degree of therapeutic efficacy with a minimum of unacceptable adverse effects. In situations where the agent's plasma concentration can be measured and related to therapeutic window, additional guidance for dosage modification can be obtained.

As used herein, the terms “treat”, “treating”, and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

### I. Methods of the Invention

The present invention is based on the discovery of stages during differentiation of pluripotent stem cells to RPE cells when RPE progenitor cells may be isolated, partially purified, and further differentiated to mature RPE cells with minimal or without manual picking of the RPE cells. Any method for differentiating pluripotent cells into RPE cells may be used. For example, RPE cells may be obtained by differentiating pluripotent stem cells through a monolayer method as described herein and also described in WO 2005/070011, which is incorporated herein by reference in its entirety. Other methods include obtaining embryoid bodies from pluripotent stem cells and differentiating the embryoid bodies into RPE cells, also described in WO 2005/070011 as well as in WO 2014/121077, which is incorporated by reference in its entirety. In another example, pluripotent stem cells may be differentiated towards the RPE cell lineage using a first differentiating agent and then further differentiated towards RPE cells using a member of the transforming factor-β (TGFβ) superfamily, as well as homologous ligands including activin (e.g., activin A, activin B, and activin AB), nodal, anti-mullerian hormone (AMH), bone morphogenetic proteins (BMP) (e.g., BMP2, BMP3, BMP4, BMP5, BMP6, and BMP7, and growth and differentiation factors (GDF)), as described in, for example, WO 2019130061, which is incorporated herein by reference in its entirety. In an embodiment, RPE cells may be obtained by (a) culturing pluripotent stem cells in a medium comprising a first differentiating agent (e.g., nicotinamide) and (b) culturing the cells obtained in step (a) in a medium comprising a member of the TGFβ superfamily (e.g., activin A) and the first differentiating agent (e.g., nicotinamide), as described in WO 2019130061. In yet another example, a single cell suspension of pluripotent stem cells may be used to differentiate into RPEs as described in WO 2017/044488, which is incorporated herein by reference in its entirety. Accordingly, the RPEs may be obtained from pluripotent stem cells in which the pluripotent stem cells are differentiated in one or more steps in one or more differentiation media that may comprise differentiation factors, such as one or more of a WNT pathway inhibitor (e.g., CKI-7, DKK1), a TGF pathway inhibitor (e.g., LDN193189), a BMP pathway inhibitor (e.g., SB431542), a MEK inhibitor (e.g., PD0325901), a member of the transforming factor-β (TGFβ) superfamily, and homologous ligands such as activin. Additionally, the RPE cells may be obtained from non-adherent or adherent cultures and from feeder or feeder-free cultures.

During the differentiation process when there is a sufficient number of clusters of RPE progenitor cells (e.g., identified as PAX6/MITF positive cells) that stay together, the clusters of RPE progenitor cells may be treated with a dissociation reagent, followed by size fractionation of the clusters and subsequent subculture of the RPE progenitor cells as single cells or cell clusters to produce RPE cells. The methods of the invention are both simple and efficient, and result in cultures of RPE cells that are, in some embodiments, substantially pure.

In an aspect, the present invention provides a method for producing a population of retinal epithelium (RPE) cells, the method comprising: (i) obtaining cell clusters of PAX6+/MITF+RPE progenitor cells and dissociating the cell clusters into single cells; (ii) culturing the single cells in a differentiation medium such that the cells differentiate to RPE cells; and (iii) harvesting the RPE cells produced in step (ii); thereby producing a population of RPE cells. In another aspect, the present invention provides a method for producing a population of retinal epithelium (RPE) cells, the method comprising: (i) obtaining cell clusters of PAX6+/MITF+RPE progenitor cells, (ii) culturing the cell clusters in a differentiation medium such that the cells differentiate to RPE cells; and (iii) harvesting the RPE cells produced in step (ii); thereby producing a population of RPE cells. In any of the embodiments of the present invention, the PAX6+/MITF+RPE progenitor cells may be obtained from a population of pluripotent stem cells.

In an aspect, the present invention provides a method for producing a population of retinal epithelium (RPE) cells, the method comprising: (i) culturing a population of pluripotent stem cells in a first differentiation medium, such that the cells differentiate into RPE progenitor cells; (ii) dissociating the RPE progenitor cells, fractionating the cells to collect RPE progenitor cell clusters, dissociating the RPE progenitor cell clusters into single cells, and subculturing the single cells in a second differentiation medium such that the cells differentiate to RPE cells; and (iii) harvesting the RPE cells produced in step (ii); thereby producing a population of RPE cells. In another aspect, the present invention provides a method for producing a population of retinal epithelium (RPE) cells, the method comprising: (i) culturing a population of pluripotent stem cells in a first differentiation medium, such that the cells differentiate into RPE progenitor cells; (ii) dissociating the RPE progenitor cells, fractionating the cells to collect RPE progenitor cell clusters, and subculturing the collected RPE progenitor cell clusters in a second differentiation medium such that the cells differentiate to RPE cells; and (iii) harvesting the RPE cells produced in step (ii) thereby producing a population of RPE cells. In an embodiment of the present invention, the RPE progenitor cells are positive for PAX6/MITF. In another embodiment, prior to step (i), the pluripotent stem cells are cultured on feeder cells in a medium that supports pluripotency. In a further embodiment, prior to step (i), the pluripotent stem cells are cultured feeder-free in a medium that supports pluripotency. In an embodiment, the medium that supports pluripotency is supplemented with bFGF.

The methods may further comprise harvesting the RPE cells produced in step (ii) by dissociating the RPE cells, fractionating the RPE cells to collect RPE cell clusters, dissociating the RPE cell clusters into single RPE cells, and culturing the single RPE cells. In another embodiment, the method may further comprise harvesting the RPE cells produced in step (ii) by dissociating the RPE cells, collecting RPE cell clusters, and selectively picking RPE cell clusters. The method may additionally comprise dissociating the selectively picked RPE cell clusters into single RPE cells and culturing the single RPE cells.

In an embodiment, pluripotent stem cells are human pluripotent stem cells and the RPE cells are human RPE cells. Any of these steps may be performed in non-adherent or adherent cultures, and under feeder or feeder-free conditions.

In an embodiment, the RPE progenitor cell clusters and/or the RPE cell clusters have a size of between about 40 to about 200 μm, about 40 to about 100 μm, about 40 μm to about 70 μm, about 70 μm to about 100 μm, about 70 μm to about 200 μm, or about 100 μm to about 200 μm.

In some embodiments, the pluripotent stem cells are human embryonic stem cells. In other embodiments, the pluripotent stem cells are human iPS cells. In some embodiments, the RPE cells are further expanded following harvesting. In some embodiments, the methods of the invention result in cultures of RPE cells that are substantially pure. A substantially purified population of RPE cells is one in which the RPE cells comprise at least about 75% of the cells in the population. In other embodiments, a substantially purified population of RPE cells is one in which the RPE cells comprise at least about 80%, 85%, 90%, 91%, 92%, 93%, 94%, 95%, 96%, 97%, 97.5%, 98%, 99%, or even greater than 99% of the cells in the population. In any of the embodiments, the RPE cells are human RPE cells.

In any of the embodiments of the present invention, the RPE cells express one or more of markers selected from the group RPE65, CRALBP, PEDF, Bestrophin (BEST1), MITF, OTX2, PAX2, PAX6, premelanosome protein (PMEL or gp-100), tyrosinase, and ZO1. In an embodiment, the RPE cells express Bestrophin, PMEL, CRALBP, MITF, PAX6, and ZO1. In a further embodiment, the RPE cells express Bestrophin, PAX6, MITF, and RPE65. In an embodiment, the RPE cells express MITF and at least one marker selected from Bestrophin and PAX6.

In any of the embodiments of the present invention, the RPE cells lack substantial expression of one or more stem cell markers selected from the group OCT4, NANOG, REX1, alkaline phosphatase, SOX2, TDGF-1, DPPA-2, DPPA-4, stage specific embryonic antigen (SSEA)-3 and SSEA-4, tumor rejection antigen (TRA)-1-60 and TRA-1-80. In an embodiment, the RPE cells lack substantial expression of OCT4, SSEA4, TRA-1-81, and alkaline phosphatase. In another embodiment, the RPE cells lack substantial expression of OCT4, NANOG, and SOX2.

### Culturing Pluripotent Stem Cells

Pluripotent stem cells, e.g., embryonic stem (ES) cells or iPS cells, may be the starting material of the disclosed method. In any of the embodiments herein, the pluripotent stem cell may be human pluripotent stem cells (hPSCs). Pluripotent stem cells (PSCs) may be cultured in any way known in the art, such as in the presence or absence of feeder cells. Additionally, PSCs produced using any method can be used as the starting material to produce RPE cells. For example, the hES cells may be derived from blastocyst stage embryos that were the product of in vitro fertilization of egg and sperm. Alternatively, the hES cells may be derived from one or more blastomeres removed from an early cleavage stage embryo, optionally, without destroying the remainder of the embryo. In still other embodiments, the hES cells may be produced using nuclear transfer. In a further embodiment, iPSCs may be used. As a starting material, previously cryopreserved PSCs may be used. In another embodiment, PSCs that have never been cryopreserved may be used.

In one aspect of the present invention, PSCs are plated onto an extracellular matrix under feeder or feeder-free conditions. In some embodiments, the extracellular matrix is laminin with or without e-cadherin. In some embodiments, laminin may be selected from the group comprising laminin 521, laminin 511, or iMatrix511. In some embodiments, the feeder cells are human dermal fibroblasts (HDF). In other embodiments, the feeder cells are mouse embryo fibroblasts (MEF).

In certain embodiments, the media used when culturing the PSCs may be selected from any media appropriate for culturing PSCs. In some embodiments, any media that is capable of supporting PSC cultures may be used. For example, one of skill in the art may select amongst commercially available or proprietary media. In further embodiments, the PSCs can be cultured on an extracellular matrix, including, but not limited to, laminin, fibronectin, vitronectin, Matrigel, CellStart, collagen, or gelatin in a medium that supports pluripotency.

The medium that supports pluripotency may be any such medium known in the art. In some embodiments, the medium that supports pluripotency is Nutristem™. In some embodiments, the medium that supports pluripotency is TeSR™. In some embodiments, the medium that supports pluripotency is StemFit™. In other embodiments, the medium that supports pluripotency is Knockout™ DMEM (Gibco), which may be supplemented with Knockout™ Serum Replacement (Gibco), LIF, bFGF, or any other factors. Each of these exemplary media is known in the art and commercially available. In further embodiments, the medium that supports pluripotency may be supplemented with bFGF or any other factors. In an embodiment, bFGF may be supplemented at a low concentration (e.g., 4 ng/mL). In another embodiment, bFGF may be supplemented at a higher concentration (e.g., 100 ng/mL), which may prime the PSCs for differentiation.

The concentration of PSCs to be used in the production method of the present invention is not particularly limited. For example, when a 10 cm dish is used, 1×10⁴-1×10⁸ cells per dish, preferably 5×10⁴-5×10⁶ cells per dish, more preferably 1×10⁵-1×10⁷ cells, per dish are used.

In some embodiments, the PSCs are plated with a cell density of about 1,000-100,000 cells/cm². In some embodiments, the PSCs are plated with a cell density of about 5000-100,000 cells/cm², about 5000-50,000 cells/cm², or about 5000-15,000 cells/cm². In other embodiments, the PSCs are plated at a density of about 10,000 cells/cm².

In some embodiments, the medium that supports pluripotency, e.g., StemFit™ or other similar medium, is replaced with a differentiation medium (e.g., a medium without pluripotency-supporting factors such as bFGF) to differentiate the cells into RPE cells. In an embodiment, embryoid bodies (EBs) are formed from the PSCs and the EBs are further differentiated into RPE cells.

In some embodiments, replacement of the media from the medium that supports pluripotency to a differentiation medium may be performed at different time points during the cell culture of PSCs and may also depend on the initial plating density of the PSCs. In some embodiments, replacement of the media can be performed after 3-14 days of culture of the PSCs in the pluripotency medium. In some embodiments, replacement of the media may be performed at day 3, 4, 5, 6, 7, 8, 9, 10, 11, 12, 13, or 14.

### Differentiation of Pluripotent Stem Cells

Differentiation of pluripotent stem cells to RPE cells is initiated following replacement of the medium that supports pluripotency with one or more differentiation medium, e.g., EBDM. In some embodiments, the pluripotent stem cells are spontaneously differentiated into RPE cells in the absence of differentiation-inducing factors. In other embodiments, differentiation-inducing factors such as activin, a nodal signal inhibitor, a Wnt signal inhibitor, or a sonic hedgehog signal inhibitor may be used to differentiate pluripotent stem cells into RPE cells.

In some embodiments, the differentiation medium is EB differentiation medium (EBDM). EBDM comprises Knockout™ DMEM (Gibco) with Xeno-free KnockOut™ Serum Replacement (XF-KSR) (Gibco), beta-mercaptoethanol, NEAA, and glutamine. Any other differentiation medium known in the art may be used. In another embodiment, the differentiation medium may comprise one or more differentiation agents, such as nicotinamide, a member of the transforming factor-β (TGFβ) superfamily (e.g., activin A, activin B, and activin AB), nodal, anti-mullerian hormone (AMH), bone morphogenetic proteins (BMP) (e.g., BMP2, BMP3, BMP4, BMP5, BMP6, and BMP7, growth and differentiation factors (GDF)), WNT pathway inhibitor (e.g., CKI-7, DKK1), a TGF pathway inhibitor (e.g., LDN193189, Noggin), a BMP pathway inhibitor (e.g., SB431542), a sonic hedgehog signal inhibitor, a bFGF inhibitor, and/or a MEK inhibitor (e.g., PD0325901). In an embodiment, the pluripotent stem cells may be differentiated towards the RPE cell lineage in a first differentiation medium comprising a first differentiation agent and then further differentiated towards RPE cells in a second differentiation medium comprising a second differentiation agent. In an embodiment, the first differentiation medium comprises nicotinamide and the second differentiation medium comprises activin (e.g., activin A). Additionally, the RPE cells may be obtained from non-adherent or adherent cultures, and under feeder or feeder-free conditions.

In an embodiment, the differentiation media may be changed every day during differentiation. In some embodiments, the differentiation media is subsequently changed every 2-3 days during differentiation. In some embodiments, the cells are cultured in differentiation media for about 3-12 weeks, e.g., 6-10 weeks, 2-8 weeks, or 3-6 weeks.

In an embodiment, following replacement of the medium that supports pluripotency with a differentiation medium, molecular markers and morphological features may be detected in order to determine differentiation of pluripotent cells and identify RPE progenitor cells in culture. Whether or not a cell is an RPE cell or an RPE progenitor may be judged by changes in cell morphology (e.g., intracellular melanin pigment deposition, polygonal and flat cell morphology, formation of polygonal actin bundle, etc.) as an index by using an optical or electron microscope. Detection of molecular, morphological, and other features of RPEs are described, for example, in U.S. Pat. Nos. 7,794,704; 7,736,896; WO 2009/051671; WO 2012/012803; WO 2013/074681; WO 2011/063005; and WO 2016/154357, incorporated in their entireties herein by reference. Accordingly, in some embodiments, after the medium that supports pluripotency is replaced with a differentiation medium, the differentiation of pluripotent cells is observed by the identification of morphological features of the RPE progenitor cells in culture.

In further embodiments, after the medium that supports pluripotency is replaced with a differentiation medium, the differentiation of pluripotent cells is identified by observing the changes in gene expression of the molecular markers of differentiated cells. In some embodiments, the molecular markers of differentiated cells are upregulated. In further embodiments, the molecular markers of pluripotency are downregulated. In some embodiments, the changes in gene expression of the molecular markers of differentiated cells can be confirmed by qPCR/scorecard and/or by immunostaining. In some embodiments, the changes in gene expression of the molecular markers of differentiated cells are observed after about three weeks of differentiation.

In some embodiments, a molecular marker of retinal lineage is PAX6, and a marker of pigmented cells is MITF. Therefore, a population of cells expressing PAX6 and/or MITF indicate that the progenitors of retinal lineage/RPE are present and can be isolated from the culture.

In other embodiments, it may not be necessary to determine differentiation of pluripotent cells and identify RPE progenitors as long as the culture conditions are known to produce RPE progenitor cells. Thus, PAX6 and MITF-positive clusters may be isolated without having to test for PAX6/MITF.

### Isolation and Subculture of RPE Progenitor Cells

The cells of epithelial morphology are held together in culture by formation of tight junctions and generate clusters of similar type of cells during differentiation. Thus, in some embodiments, for isolation of the desired RPE progenitor cell population, the differentiating culture is digested or dissociated, e.g., with an enzymatic or non-enzymatic dissociation reagent, e.g., a collagenase or dispase, to form a suspension containing cellular clusters comprising RPE progenitor cells and single cells. Single cells and non-epithelial cells may be separated and discarded as described below. Additionally, large clusters of non-RPE cells as well as clusters containing a mixture of RPEs and non-RPEs may be eliminated by size fractionation as described below, allowing for increased purity.

In some embodiments, to isolate the desired RPE progenitor cell population, the differentiating culture can be digested with a dissociation reagent and allow for isolation of free floating clusters of cells. In some embodiments, the dissociation reagent is collagenase. In other embodiments, the dissociation reagent is dispase. In some embodiments, the dissociation with the dissociation reagent is carried out overnight. In some embodiments, the dissociation with the dissociation reagent is carried out for about 2-30 hours. In an embodiment, the dissociation with the dissociation reagent is carried out for about 3-10 hrs or about 3-6 hrs. In an embodiment, the dissociation with the dissociation reagent is carried out for about 3, 4, 5, 6, 7, 8, 9, 10, 11, 12, 13, 14, 15, 16, 17, or 18 hours.

In some embodiments, dissociation is performed at about 2 to 12 weeks after onset of differentiation. In some embodiments, dissociation is performed at about 2 weeks, about 3 weeks, 4 weeks, about 5 weeks, about 6 weeks, about 7 weeks, about 8 weeks, about 9 weeks, about 10 weeks, about 11 weeks or about 12 weeks after onset of differentiation. In further embodiments, dissociation is performed on clusters of epithelial morphology positive for PAX6 and MITF.

In another aspect of the methods disclosed herein, in order to isolate RPE progenitor cell clusters, the suspension containing cellular clusters and single cells are fractionated. Any method for collecting the desired RPE progenitor cell clusters may be used. In an embodiment, single cells and other undesirable cells may be passed through a cell strainer or a series of cell strainers and the desired cell cluster populations may be collected by harvesting the cells remaining on the cell strainer. In some embodiments, the cell clusters collected for further processing comprise cell clusters of between about 40 μm and about 100 μm in size. In other embodiments, the collected cell clusters comprise cell clusters of between about 40 μm and about 200 μm in size. In some embodiments, the collected cell clusters comprise cell clusters of about 40 μm in size. In some embodiments, the collected cell clusters comprise cell clusters of about 50 μm in size. In some embodiments, the collected cell clusters comprise cell clusters of about 60 μm in size. In some embodiments, the collected cell clusters comprise cell clusters of about 70 μm in size. In some embodiments, the collected cell clusters comprise cell clusters of about 80 μm in size. In some embodiments, the collected cell clusters comprise cell clusters of about 90 μm in size. In some embodiments, the collected cell clusters comprise cell clusters of about 100 μm in size. In some embodiments, the collected cell clusters comprise cell clusters of about 110 μm in size. In some embodiments, the collected cell clusters comprise cell clusters of about 120 μm in size. In some embodiments, the collected cell clusters comprise cell clusters of about 130 μm in size. In some embodiments, the collected cell clusters comprise cell clusters of about 140 μm in size. In some embodiments, the collected cell clusters comprise cell clusters of about 150 μm in size. In some embodiments, the collected cell clusters comprise cell clusters of about 160 μm in size. In some embodiments, the collected cell clusters comprise cell clusters of about 170 μm in size. In some embodiments, the collected cell clusters comprise cell clusters of about 180 μm in size. In some embodiments, the collected cell clusters comprise cell clusters of about 190 μm in size. In some embodiments, the collected cell clusters comprise cell clusters of about 200 μm in size.

In some embodiments, single cells and cell cultures that do not meet the desired size requirement are discarded. In some embodiments, a series of cell strainers may be used to collect cell clusters having the desired size requirements. For instance, the first cell strainer may have a low mesh size (e.g., 40 μm) and the cell cluster population that remains on the first cell strainer are collected. The collected cell cluster population may then be placed on a second cell strainer having a higher mesh size (e.g., 200 μm, 100 μm), and the cell cluster population that pass through the second cell strainer may be collected to obtain the desired size requirement (e.g., 40 μm-200 μm or 40 μm-100 μm). Alternatively, the first cell strainer may be a first cell strainer with a higher mesh size (e.g., 200 μm, 100 μm) such that the cell cluster population that passes through the cell strainer is collected and larger cell clusters remaining on the first cell strainer are discarded. The pass-through cells may then be placed on a second cell strainer having a smaller mesh size (e.g., 40 μm) such that the cell clusters remaining on the second cell strainer are collected and have the desired size requirement (e.g., 40 μm-200 μm or 40 μm-100 μm).

The collected RPE progenitor cells may be subcultured as clusters or as single cells to obtain proliferating and mature RPE cells according to the methods described below.

### Single RPE Progenitor Cell Subculture Method for Obtaining RPE Cells

In the single RPE progenitor cell subculture method, the RPE progenitor cell clusters obtained as described above may be dissociated with a dissociation reagent to obtain single cells, and the population of RPE progenitor single cells are subcultured in a differentiation medium until RPE cells are obtained. In an embodiment, the cells are subcultured on laminin, e.g., laminin 521, laminin 511, or iMatrix511, or other extracellular matrix, such as, fibronectin, vitronectin, Matrigel, CellStart, collagen, or gelatin. In some embodiments, the cells are subcultured for about 1 to 8 weeks. In some embodiments, the cells are subcultured for about 2 weeks, 3, weeks, 4 weeks, 5 weeks, 6 weeks, 7 weeks, or 8 weeks. In other embodiments, the cells are subcultured for at least 8 weeks. In an embodiment, the cells may be subcultured under adherent conditions, such as on an adherent culture dish. In another embodiment, the cells may be subcultured under non-adherent conditions, and under feeder or feeder-free conditions.

The RPE cells may then be harvested, for example, with a dissociation reagent and obtaining RPE cell clusters. RPE cell clusters may be obtained by harvesting the RPE cells and removing single cells by any method known in the art. In an embodiment, the RPE cells may be harvested and passed through a strainer or a series of strainers as described above, to obtain RPE cell clusters. Any cell strainer size may be used, for example, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm in size, or a combination thereof. The RPE cell clusters obtained may be at least 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm in size. In some embodiments, the RPE cell clusters collected for further processing comprise cell clusters of about 40 μm and about 100 μm in size. In other embodiments, the collected RPE cell clusters comprise cell clusters of about 40 μm and about 200 μm in size. In some embodiments, the collected RPE cell clusters comprise cell clusters of about 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm in size.

In an embodiment, the RPE cell clusters obtained may be dissociated into single cells with an enzymatic or non-enzymatic dissociation reagent and cultured to expand the RPE cells, further described below.

In an alternative embodiment, islands of pigmented cells may be selectively picked from the RPE cell clusters obtained. This selective/minimal picking process is substantially easier with the desirable cell population having been concentrated in the prior subculturing step, resulting in a high purity of RPEs. The RPEs may be selectively picked manually, e.g. mechanically using a glass capillary, by using an optical microscope, etc., or by an automated system that can recognize RPE cells from other types of cells. The selected RPE clusters may then be dissociated to generate single RPE cells. The single RPE cells may be cultured to expand the RPE cells as further described below.

In any of the embodiments of the present invention, the RPE cells express one or more of markers selected from the group RPE65, CRALBP, PEDF, Bestrophin, MITF, OTX2, PAX2, PAX6, premelanosome protein (PMEL or gp-100), tyrosinase, and ZO1. In an embodiment, the RPE cells express Bestrophin, PMEL, CRALBP, MITF, PAX6, and ZO1. In a further embodiment, the RPE cells express Bestrophin, PAX6, MITF, and RPE65. In an embodiment, the RPE cells express MITF and at least one marker selected from Bestrophin and PAX6. In any of the embodiments of the present invention, the RPE cells lack substantial expression of one or more stem cell markers selected from the group OCT4, NANOG, REX1, alkaline phosphatase, SOX2, TDGF-1, DPPA-2, DPPA-4, stage specific embryonic antigen (SSEA)-3 and SSEA-4, tumor rejection antigen (TRA)-1-60 and TRA-1-80. In an embodiment, the RPE cells lack substantial expression of OCT4, SSEA4, TRA-1-81, and alkaline phosphatase. In another embodiment, the RPE cells lack substantial expression of OCT4, NANOG, and SOX2.

In some embodiments, a sample of the RPE cells produced may be tested for the desired molecular marker profile and then harvested. In other embodiments, it may not be necessary to test the RPE cells for molecular markers before harvesting as long as the culture conditions are known to produce RPE cells. Thus, RPE cells may be harvested without having to test for molecular markers.

### RPE Progenitor Cell Cluster Subculturing Method for Obtaining RPE Cells

In the RPE progenitor cell cluster subculturing method, the RPE progenitor cell clusters obtained after size fractionation as described above are subcultured in differentiation medium as cell clusters until RPE cells are obtained. In an embodiment, the RPE progenitor cell clusters are subcultured onto laminin, e.g., laminin 521, laminin 511, or iMatrix511, or other extracellular matrix, such as fibronectin, vitronectin, Matrigel, CellStart, collagen, or gelatin. In some embodiments, the cell clusters are subcultured for about 1 to 8 weeks. In some embodiments, the cell clusters are subcultured for about 2 weeks, 3, weeks, 4 weeks, 5 weeks, 6 weeks, 7 weeks, or 8 weeks. In other embodiments, the cell clusters are subcultured for at least 8 weeks. In an embodiment, the cell clusters may be subcultured under non-adherent conditions. In another embodiment, the cell clusters may be subcultured under adherent conditions. In another embodiment, the cell clusters may be cultured under feeder or feeder-free conditions.

The RPE cells may then be harvested, for example, with a dissociation reagent to obtain RPE cell clusters. RPE cell clusters may be obtained by harvesting the RPE cells and removing single cells by any method known in the art. In an embodiment, the RPE cells may be harvested and passed through a strainer or a series of strainers as described above, to obtain RPE cell clusters. Any cell strainer size may be used, for example, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm in size, or a combination thereof. The RPE cell clusters obtained may be at least 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm in size. In some embodiments, the RPE cell clusters collected for further processing comprise cell clusters of about 40 μm and about 100 μm in size. In other embodiments, the collected RPE cell clusters comprise cell clusters of about 40 μm and about 200 μm in size. In some embodiments, the collected RPE cell clusters comprise cell clusters of about 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm in size.

In an embodiment, the RPE cell clusters obtained may be dissociated into single cells with an enzymatic or non-enzymatic dissociation reagent and cultured to expand the RPE cells, further described below.

In an alternative embodiment, islands of pigmented cells may then be selectively picked from the RPE cell clusters obtained. This selective/minimal picking process is substantially easier with the desirable cell population having been concentrated in the prior subculturing step, resulting in a high purity of RPEs. The RPEs may be selectively picked manually, e.g., mechanically using a glass capillary, by using an optical microscope, etc., or by an automated system that can recognize RPE cells from other types of cells. The selected RPE clusters may then be dissociated to generate single RPE cells. The single RPE cells may be cultured to expand the RPE cells as further described below.

In any of the embodiments of the present invention, the RPE cells express one or more of markers selected from the group RPE65, CRALBP, PEDF, Bestrophin, MITF, OTX2, PAX2, PAX6, premelanosome protein (PMEL or gp-100), tyrosinase, and ZO1. In an embodiment, the RPE cells express Bestrophin, PMEL, CRALBP, MITF, PAX6, and ZO1. In a further embodiment, the RPE cells express Bestrophin, PAX6, MITF, and RPE65. In an embodiment, the RPE cells express MITF and at least one marker selected from Bestrophin and PAX6. In any of the embodiments of the present invention, the RPE cells lack substantial expression of one or more stem cell markers selected from the group OCT4, NANOG, REX1, alkaline phosphatase, SOX2, TDGF-1, DPPA-2, DPPA-4, stage specific embryonic antigen (SSEA)-3 and SSEA-4, tumor rejection antigen (TRA)-1-60 and TRA-1-80. In an embodiment, the RPE cells lack substantial expression of OCT4, SSEA4, TRA-1-81, and alkaline phosphatase. In another embodiment, the RPE cells lack substantial expression of OCT4, NANOG, and SOX2.

In some embodiments, a sample of the RPE cells produced may be tested for the desired molecular marker profile and then harvested. In other embodiments, it may not be necessary to test the RPE cells for molecular markers before harvesting as long as the culture conditions are known to produce RPE cells. Thus, RPE cells may be harvested without having to test for molecular markers.

### Expansion of RPE Cells

In some embodiments, the RPE cells obtained from the single RPE progenitor cell subculture or RPE progenitor cell cluster subculture method may be cultured onto an extracellular matrix, such as laminin, fibronectin, vitronectin, Matrigel, CellStart, collagen, or gelatin, in a medium that supports RPE growth or proliferation to expand the RPE cell population.

The RPE cell population first cultured in this step is referred to herein as “P0.” In an embodiment, the extracellular matrix is selected from the group consisting of laminin, fibronectin, vitronectin, Matrigel, CellStart, collagen, and gelatin. In some embodiments, the extracellular matrix is laminin. In an embodiment, the laminin is selected from laminin 521, laminin 511, or iMatrix511. In further embodiments, laminin comprises e-cadherin. In another embodiment, the extracellular matrix is gelatin. In some embodiments, the medium is RPE-MM (also referred to as RPEGMMM, MM or maintenance medium and comprising DMEM/KO-DMEM with KSR and FBS, beta-mercaptoethanol, NEAA, and glutamine), StemFit, EGM2, or EBDM. In some embodiments, the RPE-MM is supplemented with FGF (MM/FGF). In other embodiments, other medium known in the art that supports RPE growth and expansion may be used. Any such medium may be supplemented with or without FBS and/or bFGF, or any other factors, such as heparin, hydrocortisone, vascular endothelial growth factor, recombinant insulin-like growth factor, ascorbic acid, or human epidermal growth factor. See e.g., WO2013074681A, which is incorporated herein by reference in its entirety.

In an embodiment, the RPE cells may be passaged and cultured until adequate numbers of RPE cells are obtained. In an embodiment, the RPE cells are passaged indefinitely. In another embodiment, the RPE cells are passaged at least one time (“P1”) up to 20 times (“P20”). In an embodiment, the RPE cells are passaged at least two times (“P2”) up to 8 times (“P8”). In a further embodiment, the RPE cells are passaged two times (“P2”), three times (“P3”), four times, (“P4”), five times (“P5”), six times (“P6”), seven times (“P7”), or eight times (“P8”). The RPE cells may be cryopreserved until further use. In an embodiment, the duration of each expansion phase may vary from days, weeks, to months. In an embodiment, the duration of the expansion phase is between about 2-90 days. In another embodiment, the duration of the expansion phase is between about 2-60 days, 3-50 days, 3-40 days, 3-30 days, 3-25 days, 8-25 days, 10-25 days, or 2-14 days, or 2-10 days. During the expansion phase, fresh medium may be added at intervals, such as every 1-2 days. In an embodiment, bFGF is added at a concentration of about 1-100 ng/ml to the RPE cell culture medium during the first 1-5 days, 1-4 days, 1-3 days, 1-2 days, 1 day, 2 days, 3 days, 4 days, or 5 days of RPE expansion at each passage (e.g., P0, P1, P2) and then removed until further passaged. In an embodiment, the bFGF concentration is about 1-50 ng/ml, about 2-40 ng/ml, about 3-30 ng/ml, about 4-20 ng/ml, or about 4-10 ng/ml. In a specific embodiment, the bFGF concentration is about 4 ng/ml, 5 ng/ml, 6 ng/ml, 7 ng/ml, 8 ng/ml, 9 ng/ml, or 10 ng/ml.

In any of the embodiments of the present invention, the RPE cells express one or more of markers selected from the group RPE65, CRALBP, PEDF, Bestrophin, MITF, OTX2, PAX2, PAX6, premelanosome protein (PMEL or gp-100), tyrosinase, and ZO1. In an embodiment, the RPE cells express Bestrophin, PMEL, CRALBP, MITF, PAX6, and ZO1. In a further embodiment, the RPE cells express Bestrophin, PAX6, MITF, and RPE65. In an embodiment, the RPE cells express MITF and at least one marker selected from Bestrophin and PAX6. In any of the embodiments of the present invention, the RPE cells lack substantial expression of one or more stem cell markers selected from the group OCT4, NANOG, REX1, alkaline phosphatase, SOX2, TDGF-1, DPPA-2, DPPA-4, stage specific embryonic antigen (SSEA)-3 and SSEA-4, tumor rejection antigen (TRA)-1-60 and TRA-1-80. In an embodiment, the RPE cells lack substantial expression of OCT4, SSEA4, TRA-1-81, and alkaline phosphatase. In another embodiment, the RPE cells lack substantial expression of OCT4, NANOG, and SOX2.

In some embodiments, a sample of the RPE cells produced may be tested for the desired molecular marker profile and then harvested. In other embodiments, it may not be necessary to test the RPE cells for molecular markers before harvesting as long as the culture conditions are known to produce RPE cells. Thus, RPE cells may be harvested without having to test for molecular markers.

### Feeder and Feeder-Free Based Cultures

### Mouse Feeder Layers

The PSCs, as disclosed herein, may be cultured on mouse embryonic fibroblasts (MEF) as a feeder cell (see, e.g., Thomson J A, Itskovitz-Eldor J, Shapiro S S, Waknitz M A, Swiergiel J J, Marshall V S, Jones J M. (1998); Science 282: 1145-7; Reubinoff B E, Pera M F, Fong C, Trounson A, Bongso A. (2000); Reubinoff et al., 2000, Nat. Biotechnol. 18: 399-404). MEF cells may be derived from day 12-13 mouse embryos in medium supplemented with fetal bovine serum.

PSCs may be cultured on MEF under serum-free conditions using serum replacement supplemented with basic fibroblast growth factor (bFGF) (see, e.g., Amit M, Carpenter M K, Inokuma M S, Chiu C P, Harris C P, Waknitz M A, Itskovitz-Eldor J, Thomson J A. (2000)). Clonally derived human embryonic stem cell lines maintain pluripotency and proliferative potential for prolonged periods of culture (see, e.g., Dev. Biol. 227: 271-8). In addition, following 6 months of culturing under serum replacement the PSCs may still maintain their pluripotency when cultured under conditions that promote maintenance of the pluripotent state. The pluripotency of PSCs may be indicated by their ability to form teratomas which contain all three embryonic germ layers. Additionally, the differentiation of PSCs to RPEs may be performed in the presence of mouse feeder cells. Accordingly, the PSCs used in the methods described herein may be cultured on mouse feeder cells.

### Human Feeder Cells

PSCs may be cultured, maintained, or differentiated on human feeder cells, as described in, for example, PCT publication No. WO2009048675. PSCs may be maintained in the undifferentiated state by multiple sequential passages of the PSCs on human feeder cells (see, e.g., Richards et al., 2002, Nat. Biotechnol. 20: 933-6). PSCs may also be differentiated to RPEs in the presence of human feeder cells. Accordingly, the PSCs used in the methods described herein can be cultured on human feeder cells.

### Feeder-Free Cultures

PSCs may be cultured in a feeder-free system on a solid surface such as an extracellular matrix (e.g., Matrigel® or laminin) in the presence of a culture medium. Various methods are known in the art to differentiate PSCs ex vivo into RPE cells, as summarized in Rowland et al., Journal Cell Physiology, 227:457-466, 2012, incorporated herein by reference. Accordingly, the PSCs used in the methods described herein may be cultured on feeder-free cultures.

### Use of FGF/bFGF and ROCK Inhibitors

In mammalian development, RPE shares the same progenitor with neural retina, the neuroepithelium of the optic vesicle. Under certain conditions, RPE can transdifferentiate into neuronal progenitors (Opas and Dziak, 1994, Dev Biol. 161(2):440-54), neurons (Chen et al., 2003, J Neurochem. 84(5):972-81; Vinores et al., 1995, Exp Eye Res. 60(6):607-19), and lens epithelium (Eguchi, 1986). One of the factors which can stimulate the change of RPE into neurons is bFGF (Opas and Dziak, 1994, Dev Biol. 161(2):440-54), a process associated with the expression of transcriptional activators normally required for the eye development, including rx/rax, chx10/vsx-2/alx, ots-1, otx-2, six3/optx, six6/optx2, mitf, and PAX6/pax2 (Fischer and Reh, 2001, Dev Neurosci. 23(4-5):268-76; Baumer et al., 2003, Development; 130(13):2903-15). It has been shown that the margins of the chick retina contain neural stem cells (Fischer and Reh, 2000; Dev Biol. 15; 220(2):197-210) and that the pigmented cells in that area, which express PAX6/mitf, can form neuronal cells in response to FGF (Fischer and Reh, 2001, Dev Neurosci. 23(4-5):268-76).

In some embodiments, the PSCs of the invention may be maintained in a pluripotent state in a culture medium that includes 1-200 ng/ml bFGF. In an embodiment, the bFGF concentration is about 1-100 ng/ml, about 2-100 ng/ml, about 3-100 ng/ml, or about 4-100 ng/ml. In a specific embodiment, the bFGF concentration is about 100 ng/ml. In some embodiments, PSCs may be differentiated into RPE cells in the presence of bFGF. In other embodiments, as discussed above and herein, RPE cells may be expanded in the presence of bFGF.

During RPE formation, the pluripotent cells may be cultured in the presence of an inhibitor of rho-associated protein kinase (ROCK). ROCK inhibitors refer to any substance that inhibits or reduces the function of Rho-associated kinase or its signaling pathway in a cell, such as a small molecule, an siRNA, a miRNA, an antisense RNA, or the like. “ROCK signaling pathway,” as used herein, may include any signal processors involved in the ROCK-related signaling pathway, such as the Rho-ROCK-Myosin II signaling pathway, its upstream signaling pathway, or its downstream signaling pathway in a cell. An exemplary ROCK inhibitor that may be used is Stemgent's Stemolecule Y-27632 (see Watanabe et al., Nat Biotechnol. 2007 June; 25(6):68 1-6). Other ROCK inhibitors include, e.g., H-1 1 52, Y-3014 1, Wf-536, HA-1077, hydroxyl-HA-1077, GSK269962A and SB-772077-B. Doe et al., J. Pharmacol. Exp. Ther., 32:89-98, 2007; Ishizaki, et al, Mol. Pharmacol., 57:976-983, 2000; Nakajima et al., Cancer Chemother. Pharmacol., 52:3 1 9-324, 2003; and Sasaki et al., Pharmacol. Ther., 93:225-232, 2002, each of which is incorporated herein by reference as if set forth in its entirety. ROCK inhibitors may be utilized with concentrations and/or culture conditions as known in the art, for example as described in US Pub. No. 2012/0276063 which is hereby incorporated by reference in its entirety. For example, the ROCK inhibitor may have a concentration of about 0.05 to about 50 microM, for example, at least or about 0.05, 0.1, 0.2, 0.5, 0.8, 1, 1.5, 2, 2.5, 5, 7.5, 10, 15, 20, 25, 30, 35, 40, 45, or 50 microM, including any range derivable therein, or any concentration effective for promoting cell growth or survival. In further embodiments, the RPE expansion culture may be further supplemented with ROCK inhibitors and/or bFGF as described by PCT publication No. WO2013074681A1; incorporated in its entirety herein by reference.

### Adherent and Non-Adherent Culture

The “adherent culture” as used in the present disclosure means culture in a state where the cells of interest are adhered to a tissue culture vessel via a cell culture substrate, e.g., laminin. Cells may also adhere to plastic that has been treated for cell adhesion (“tissue culture treated”) without any additional substrate coating.

In some embodiments, the differentiation from pluripotent stem cells to RPE cells is performed by adherent culture. Adherent culture can be performed by using a cell-adhesive culture vessel. While the cell-adhesive culture vessel is not particularly limited as long as the surface of the culture vessel is treated to improve adhesiveness to the cell, for example, a culture vessel having a coated layer containing an extracellular matrix, a synthetic polymer and the like can be used. The coated layer may be constituted with one or more kinds of components, or may be formed by a single layer or multiple layers. While the extracellular matrix is not particularly limited as long as it can form a coated layer showing adhesiveness to a pluripotent stem cell, for example, collagen, gelatin, laminin, fibronectin and the like, which can be used alone or in combination. As a commercially available product containing multiple kinds of extracellular matrices, Matrigel (BD), CELLStart (Invitrogen) and the like are available. As the synthetic polymer, biologically or chemically produced polymers can be used. For example, cationic polymers such as polylysine (poly-D-lysine, poly-L-lysine), polyornithinepolyethyleneimine (PEI), poly-N-propylacrylamide (PIPAAm) and the like are preferably used. The extracellular matrix or synthetic polymer may be biologically produced by using bacterium, cells and the like and introducing genetic modification as necessary, or chemically synthesized. In other embodiments, cells may bind to the extracellular matrix via RGD peptides, which are bound by integrin adhesion receptors found on may extracellular matrices.

In some embodiments, adherent culture may be performed on a tissue culture vessel that has not been treated with any cell culture substrate or for cell adhesion. For example, media components such as FBS, fibronectin, or vitronectin may be absorbed by the tissue culture vessel and serve as cell adhesion substrates. In other embodiments, the cells in the tissue culture vessels may secrete extracellular matrices that may also serve as cell adhesion substrates.

The “non-adherent culture” as used in the present disclosure means culture in a state where the cells of interest do not adhere or substantially do not adhere to a tissue culture vessel. Accordingly, single cells or clusters of cells in a non-adherent culture may float in culture and may be in suspension. Single cells in a non-adherent culture may form clusters or aggregates under appropriate conditions. In an embodiment, the culture vessel surface may be coated with a hydrophilic, neutrally charged coating that is covalently bound to the polystyrene vessel surface, such as the Corning® Ultra-Low Attachment Surface. The non-binding surface inhibits specific and nonspecific immobilization, forcing cells into a suspended state. The cells may also be cultured in a spinner flask (Corning) to culture cells in suspension. Other methods of culturing cells in non-adherent culture are known to those skilled in the art and may be used in the methods of the present invention.

### II. Methods of Use of Retinal Pigment Epithelium Cells

RPE cells and pharmaceutical compositions comprising RPE cells produced by the methods described herein may be used for cell-based treatments in which RPE cells are needed or would improve treatment. Methods of using RPE cells provided by the present invention for treating various conditions that may benefit from RPE cell-based therapies are described herein and, for example, in U.S. Pat. No. 10,077,424, the contents of which are hereby incorporated herein by reference. The particular treatment regimen, route of administration, and any adjuvant therapy will be tailored based on the particular condition, the severity of the condition, and the patient's overall health. Additionally, in certain embodiments, administration of RPE cells may be effective to fully restore any vision loss or other symptoms. In other embodiments, administration of RPE cells may be effective to reduce the severity of the symptoms and/or to prevent further degeneration in the patient's condition. The invention contemplates that administration of a composition comprising RPE cells can be used to treat (including reducing the severity of the symptoms, in whole or in part) any of the conditions described herein. Additionally, RPE cell administration may be used to help treat the symptoms of any injury to the endogenous RPE layer.

The invention contemplates that RPE cells, including compositions comprising RPE cells, derived using any of the methods described herein can be used in the treatment of any of the indications described herein. Further, the invention contemplates that any of the compositions comprising RPE cells described herein can be used in the treatment of any of the indications described herein. In another embodiment, the RPE cells of the invention may be administered with other therapeutic cells or agents. The RPE cells may be administered simultaneously in a combined or separate formulation, or sequentially.

In an embodiment, the present invention provides a method of treating a retinal disease or disorder. In an embodiment, the retinal disease or disorder includes, for example, retinal degeneration, such as choroideremia, diabetic retinopathy, age-related macular degeneration (dry or wet), retinal detachment, retinitis pigmentosa, Stargardt's Disease, Angioid streaks, or Myopic Macular Degeneration) or glaucoma. In certain embodiments, the RPE cells of the invention may be used to treat disorders of the central nervous system, such as Parkinson's disease.

Retinitis pigmentosa is a hereditary condition in which the vision receptors are gradually destroyed through abnormal genetic programming. Some forms cause total blindness at relatively young ages, where other forms demonstrate characteristic “bone spicule” retinal changes with little vision destruction. This disease affects some 1.5 million people worldwide. Some gene defects that cause autosomal recessive retinitis pigmentosa have been found in genes expressed exclusively in RPE. One is due to an RPE protein involved in vitamin A metabolism (cis retinaldehyde binding protein (CRLBP)). Another involves a protein unique to RPE, RPE65. Mutations in the MER proto-oncogene, tyrosine kinase (MERTK) gene have also been associated with disruption of the RPE phagocytosis pathway and onset of autosomal recessive retinitis pigmentosa. Other gene defects and RPE-related retinitis pigmentosa forms are known. See e.g., Verbakel et al., Progress in Retinal and Eye Research 66:157-186 (2018). This invention provides methods and compositions for treating any or all forms of RPE-related retinitis pigmentosa by administration of RPE cells.

Animal models of retinitis pigmentosa that may be treated or used to test the efficacy of the RPE cells produced using the methods described herein include rodents (rd mouse, RPE-65 knockout mouse, tubby-like mouse, LRAT mouse, RCS rat), cats (Abyssinian cat), and dogs (cone degeneration “cd” dog, progressive rod-cone degeneration “prcd” dog, early retinal degeneration “erd” dog, rod-cone dysplasia 1, 2 & 3 “rcd1, rcd2 & rcd3” dogs, photoreceptor dysplasia “pd” dog, and Briard “RPE-65” (dog)).

In another embodiment, the present invention provides methods and compositions for treating disorders associated with retinal degeneration, including macular degeneration.

A further aspect of the present invention is the use of RPE cells for the therapy of eye diseases, including hereditary and acquired eye diseases. Examples of acquired or hereditary eye diseases are age-related macular degeneration, glaucoma and diabetic retinopathy.

Age-related macular degeneration (AMD) is the most common reason for legal blindness in Western countries. Atrophy of the submacular retinal pigment epithelium and the development of choroidal neovascularizations (CNV) results secondarily in loss of central visual acuity. For the majority of patients with subfoveal CNV and geographic atrophy there is at present no treatment available to prevent loss of central visual acuity. Early signs of AMD are deposits (drusen) between retinal pigment epithelium and Bruch's membrane. During the disease there is sprouting of choroid vessels into the subretinal space of the macula. This leads to loss of central vision and reading ability.

Glaucoma is the name given to a group of diseases in which the pressure in the eye increases abnormally. This leads to restrictions of the visual field and to the general diminution in the ability to see. The most common form is primary glaucoma; two forms of this are distinguished: chronic obtuse-angle glaucoma and acute angle closure. Secondary glaucoma may be caused by infections, tumors or injuries. A third type, hereditary glaucoma, is usually derived from developmental disturbances during pregnancy. The aqueous humor in the eyeball is under a certain pressure which is necessary for the optical properties of the eye. This intraocular pressure is normally 15 to 20 millimeters of mercury and is controlled by the equilibrium between aqueous production and aqueous outflow. In glaucoma, the outflow of the aqueous humor in the angle of the anterior chamber is blocked so that the pressure inside the eye rises. Glaucoma usually develops in middle or advanced age, but hereditary forms and diseases are not uncommon in children and adolescents. Although the intraocular pressure is only slightly raised and there are moreover no evident symptoms, gradual damage occurs, especially restriction of the visual field. Acute angle closure by contrast causes pain, redness, dilation of the pupils and severe disturbances of vision. The cornea becomes cloudy, and the intraocular pressure is greatly increased. As the disease progresses, the visual field becomes increasingly narrower, which can easily be detected using a perimeter, an ophthalmologic instrument. Chronic glaucoma generally responds well to locally administered medicaments which enhance aqueous outflow. Systemic active substances are sometimes given to reduce aqueous production. However, medicinal treatment is not always successful. If medicinal therapy fails, laser therapy or conventional operations are used in order to create a new outflow for the aqueous humor. Acute glaucoma is a medical emergency. If the intraocular pressure is not reduced within 24 hours, permanent damage occurs.

Diabetic retinopathy arises in cases of diabetes mellitus. It can lead to thickening of the basal membrane of the vascular endothelial cells as a result of glycosylation of proteins. It is the cause of early vascular sclerosis and the formation of capillary aneurysms. These vascular changes lead over the course of years to diabetic retinopathy. The vascular changes cause hypoperfusion of capillary regions. This leads to lipid deposits (hard exudates) and to vasoproliferation. The clinical course is variable in patients with diabetes mellitus. In age-related diabetes (type II diabetes), capillary aneurysms appear first. Thereafter, because of the impaired capillary perfusion, hard and soft exudates and dot-like hemorrhages in the retinal parenchyma appear. In later stages of diabetic retinopathy, the fatty deposits are arranged like a corona around the macula (retinitis circinata). These changes are frequently accompanied by edema at the posterior pole of the eye. If the edema involves the macula there is an acute serious deterioration in vision. The main problem in type I diabetes is the vascular proliferation in the region of the fundus of the eye. The standard therapy is laser coagulation of the affected regions of the fundus of the eye. The laser coagulation is initially performed focally in the affected areas of the retina. If the exudates persist, the area of laser coagulation is extended. The center of the retina with the site of sharpest vision, that is to say the macula and the papillomacular bundle, cannot be coagulated because the procedure would result in destruction of the parts of the retina which are most important for vision. If proliferation has already occurred, it is often necessary for the foci to be very densely pressed on the basis of the proliferation. This entails destruction of areas of the retina. The result is a corresponding loss of visual field. In type I diabetes, laser coagulation in good time is often the only chance of saving patients from blindness.

Another embodiment of the present invention is a method for the derivation of RPE cells or precursors to RPE cells that have an increased ability to prevent neovascularization. Alternatively such cells may be genetically modified with exogenous genes that inhibit neovascularization.

The invention contemplates that compositions of RPE cells obtained from human pluripotent stem cells (e.g., human embryonic stem cells or other pluripotent stem cells) can be used to treat any of the foregoing diseases or conditions, as well as injuries of the endogenous RPE layer. These diseases can be treated with compositions of RPE cells comprising RPE cells of varying levels of maturity, as well as with compositions of RPE cells that are enriched for mature RPE cells.

### III. Methods of Administration of Retinal Pigment Epithelium Cells

RPE cells of the invention may be administered by any route of administration appropriate for the disease or disorder being treated. In an embodiment, the RPE cells of the invention may be administered topically, systemically, or locally, such as by injection (e.g., subretinal injection), or as part of a device or implant (e.g., a sustained release implant). For example, the RPE cells of the present invention may be transplanted into the subretinal space by using vitrectomy surgery when treating a patient with a retinal disorder or disease, such as macular degeneration, Stargardt's disease, and retinitis pigmentosa. In another example, the RPE cells of the present invention may be transplanted systemically or locally when treating a patient with a CNS disorder, such as Parkinson's disease. One skilled in the art would be able to determine the route of administration for the disease or disorder being treated.

RPE cells of the invention may be delivered in a pharmaceutically acceptable ophthalmic formulation by intraocular injection, more specifically, subretinally. Concentrations for injections may be at any amount that is effective and non-toxic, depending upon the factors described herein. In some embodiments, RPE cells for treatment of a patient are formulated at doses of about 5 cells/1500 to 1×10⁷ cells/1500, 50 cells/1500 to 1×10⁶ cells/1500, or 50 cells/1500 to 5×10⁵ cells/1500. In other embodiments, RPE cells for treatment of a patient are formulated at doses of about 10, 50, 100, 500, 5000, 1×10⁴, 5×10⁴, 1×10⁵, 5×10⁵, or 1×10⁶ cells/1500. In an embodiment, about 50,000-500,000 cells may be administered to a patient. In a specific embodiment, about 50,000, 100,000, 150,000, 200,000, 250,000, 300,000, 350,000, 400,000, 450,000 or 500,000 RPE cells may be administered to a patient.

RPE cells may be formulated for delivery in a pharmaceutically acceptable ophthalmic vehicle, such that the composition is maintained in contact with the ocular surface for a sufficient time period to allow the cells to penetrate the affected regions of the eye, as for example, the anterior chamber, posterior chamber, vitreous body, aqueous humor, vitreous humor, cornea, iris/ciliary, lens, choroid, retina, sclera, suprachoridal space, conjunctiva, subconjunctival space, episcleral space, intracorneal space, epicorneal space, pars plana, surgically-induced avascular regions, or the macula. Products and systems, such as delivery vehicles, comprising the agents of the invention, especially those formulated as pharmaceutical compositions—as well as kits comprising such delivery vehicles and/or systems—are also envisioned as being part of the present invention.

In certain embodiments, a therapeutic method of the invention includes the step of administering RPE cells of the invention with an implant or device. In certain embodiments, the device is bioerodible implant for treating a medical condition of the eye comprising an active agent dispersed within a biodegradable polymer matrix, wherein at least about 75% of the particles of the active agent have a diameter of less than about 10 um. The bioerodible implant is sized for implantation in an ocular region. The ocular region can be any one or more of the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina. The biodegradable polymer can be, for example, a poly(lactic-co-glycolic)acid (PLGA) copolymer. In certain embodiments, the ratio of lactic to glycolic acid monomers in the polymer is about 25/75, 40/60, 50/50, 60/40, 75/25 weight percentage, more preferably about 50/50. Additionally, the PLGA copolymer can be about 20, 30, 40, 50, 60, 70, 80 to about 90 percent by weight of the bioerodible implant. In certain preferred embodiments, the PLGA copolymer can be from about 30 to about 50 percent by weight, preferably about 40 percent by weight of the bioerodible implant.

The volume of composition administered according to the methods described herein is also dependent on factors such as the mode of administration, number of RPE cells, age of the patient, and type and severity of the disease being treated. If administered by injection, the liquid volume comprising a composition of the invention may be from about 5.0 microliters to about 50 microliters, from about 50 microliters to about 250 microliters, from about 250 microliters to about 1 milliliter. In an embodiment, the volume for injection may be about 150 microliters.

If administered by intraocular injection, RPE cells can be delivered one or more times periodically throughout the life of a patient. For example RPE cells can be delivered once per year, once every 6-12 months, once every 3-6 months, once every 1-3 months, or once every 1-4 weeks. Alternatively, more frequent administration may be desirable for certain conditions or disorders. If administered by an implant or device, RPE cells can be administered one time, or one or more times periodically throughout the lifetime of the patient, as necessary for the particular patient and disorder or condition being treated. Similarly contemplated is a therapeutic regimen that changes over time. In certain embodiments, patients are also administered immunosuppressive therapy, either before, concurrently with, or after administration of the RPE cells. Immunosuppressive therapy may be necessary throughout the life of the patient, or for a shorter period of time. Examples of immunosuppressive therapy include, but are not limited to, one or more of: anti-lymphocyte globulin (ALG) polyclonal antibody, anti-thymocyte globulin (ATG) polyclonal antibody, azathioprine, BASILIXIMAB® (anti-IL-2Ra receptor antibody), cyclosporin (cyclosporin A), DACLIZUMAB® (anti-IL-2Ra receptor antibody), everolimus, mycophenolic acid, RITUX1MAB® (anti-CD20 antibody), sirolimus, tacrolimus (Prograf™), and mycophemolate mofetil (MMF).

In certain embodiments, RPE cells of the present invention are formulated with a pharmaceutically acceptable carrier. For example, RPE cells may be administered alone or as a component of a pharmaceutical formulation. The subject compounds may be formulated for administration in any convenient way for use in human medicine. In certain embodiments, pharmaceutical compositions suitable for parenteral administration may comprise the RPE cells, in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof. Proper fluidity can be maintained, for example, by the use of coating materials, such as lecithin, by the maintenance of the required particle size in the case of dispersions, and by the use of surfactants.

In an embodiment, the RPE cells of the present invention are formulated in GS2, which is described in WO 2017/031312, and which is hereby incorporated by reference in its entirety.

The contents disclosed in any publication cited in the present specification, including patents and patent applications, are hereby incorporated in their entireties by reference, to the extent that they have been disclosed herein.

### EXAMPLES

The following Examples are merely illustrative and are not intended to limit the scope or content of the disclosure in any way.

### Example 1: Time Course of PAX6/MITF Expression in RPE Progenitor Cells

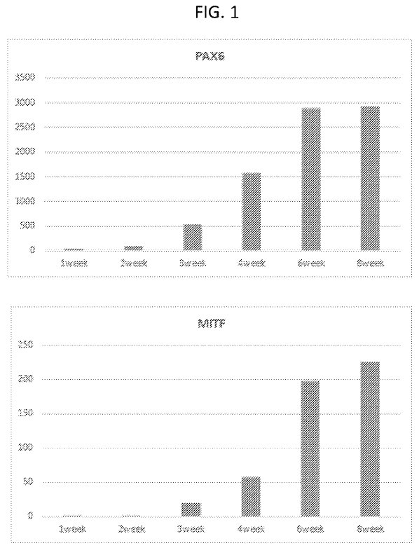

J1 hES cells were plated on laminin 521/e-cadherin-coated plates with Mitomycin C-inactivated HDF in EBDM to initiate differentiation of the J1 cells. Cells in culture were harvested at approximately 1, 2, 3, 4, 6, and 8 weeks after initiation of culture in EBDM and assessed for PAX6 and MITF expression by qPCR. As shown in FIG. 1, PAX6+/MITF+RPE progenitor cells begin appearing around weeks 3-4 in culture and the mRNA expression of PAX6 and MITF in the culture increased over time (see e.g., weeks 6-8).

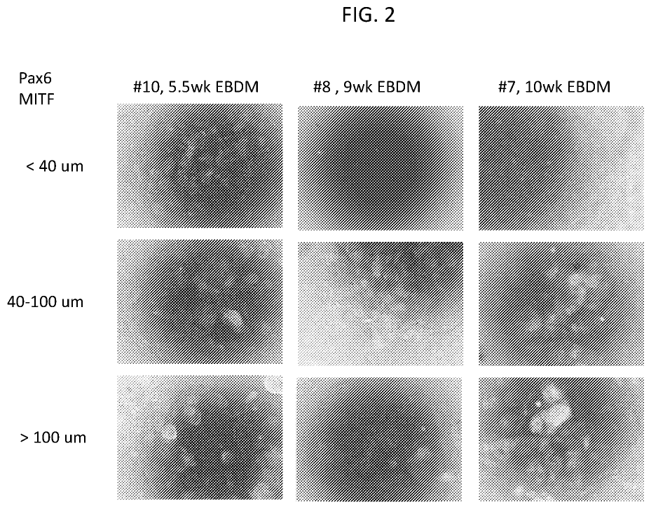

In another experiment, J1 hES cells were plated onto laminin521/e-cadherin-coated plates with Mitomycin C-inactivated HDF in Nutristem (Stemgent) for 4 days followed by TeSR2 (STEMCELL Technologies) for 8 days. The media was then switched to EBDM to initiate differentiation of the J1 cells. After approximately 5.5 weeks, 9 weeks, and 10 weeks after initiation of culture in EBDM, cells were treated with collagenase and the released digested material was passed through a column of strainers consisting of a 100 micron strainer resting atop a 40 micron strainer sitting on a collection tube. The cells that passed through the 40 micron strainer (cells that are <40 μm), cells retained on the 100 micron strainer (cells that are >100 μm), and the clusters retained on the 40 micron strainer (cells that are about 40-100 μm) were recovered and each fraction was plated onto LN521-coated wells in EBDM for three days, and the cells were fixed and stained for PAX6/MITF. As shown in FIG. 2, cells that are <40 μm showed little or no PAX6/MITF staining, even after 5.5, 9, and 10 weeks after initiation of differentiation. By 9-10 weeks after initiation of differentiation, the cells obtained from the 40-100 μm fraction showed strong PAX6/MITF staining compared to the >100 μm fraction.

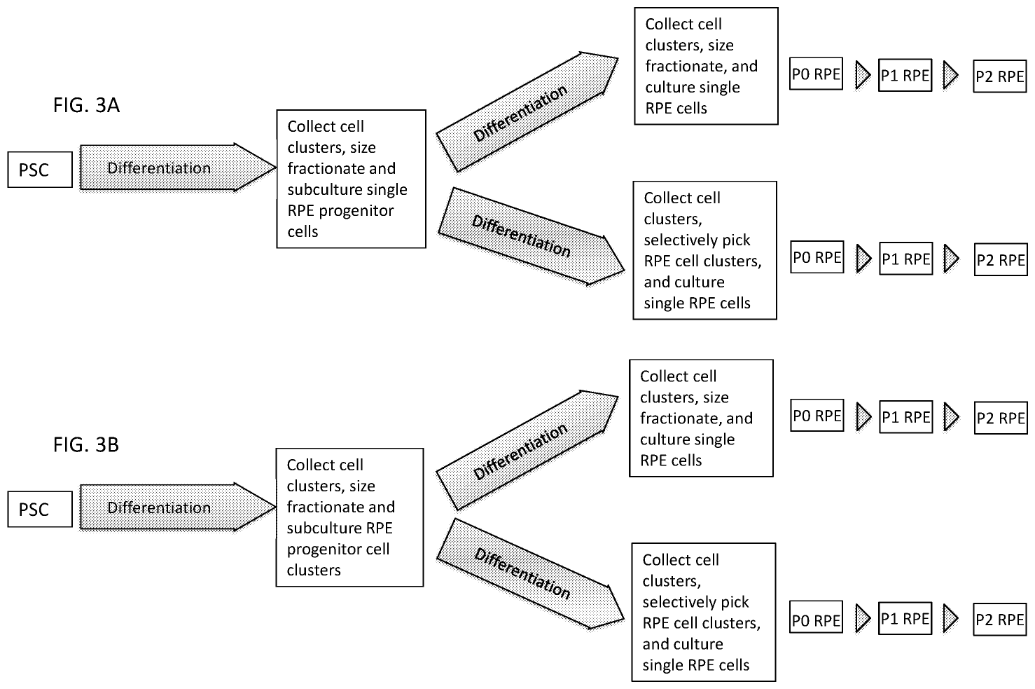

Based on these results, the timing for harvesting PAX6+/MITF+RPE progenitor cells for subculturing was identified. Exemplary processes for production of RPE cells, in accordance with some embodiments of the invention, are summarized in FIG. 3. The detailed steps of embodiments of these exemplary methods are described as follows.

### Example 2: Production of Retinal Pigment Epithelium (RPE) Cells by the Single RPE Progenitor Cell Subculture Method

In a first experiment, Mitomycin-C treated HDF cells were plated onto laminin 521/E-cadherin-coated wells. J1 hESCs were seeded onto the wells and cultured for approximately 4 days in NutriStem (Stemgent) followed by TeSR2 (STEMCELL Technologies) for 4 days. The media was then switched to EBDM to promote RPE generation and EBDM was changed every day for 7 days and then changed every 2-3 days.

After 83 days (approximately 12 weeks) in EBDM, cells were treated with collagenase overnight. The released digested material was passed through a column of strainers consisting of a 100 micron strainer resting atop a 40 micron strainer sitting on a collection tube. The clusters retained on the 40 micron strainer were recovered and dissociated into single cells by trypsin treatment for 15 min. The single cells were plated onto LN521-coated wells in EBDM and EBDM was changed every 2-3 days. After 30 days (approximately 4 weeks) in EBDM after being re-plated, cells were treated with collagenase for about 6 hrs. The released digested material was passed through a column of strainers consisting of a 100 micron strainer resting atop a 40 micron strainer sitting on a collection tube. The clusters retained on the 40 micron strainer were recovered and dissociated into single cells by 10× TrypLE (Thermo Fisher) treatment for 15 min. The single cells were plated as passage 0 RPE cells (“P0”) onto gelatin-coated wells in MM/FGF media (DMEM; GlutaMAX™-I Supplement (100×), liquid, 200 mM; FBS; KnockOut DMEM; non-essential amino acids; 2-mercaptoethanol; Knockout Serum Replacement [KSR]]+bFGF). The MM/FGF media was changed every day until about >90% confluent and then changed to MM media [the above MM/FGF media without bFGF] and fed every 2 days until harvest. P0 RPE cells were cultured for 16 days. P0 cells were harvested by 10× TrypLE treatment for 15 min and single cells were again plated as passage 1 RPE cells (“P1”) onto gelatin-coated wells in MM/FGF media. Culture method was repeated as described above for P0 RPE cells by first culturing in MM/FGF and then switching to MM media. P1 RPE cells were cultured for 14 days. P1 RPE cells were harvested and replated as passage 2 RPE cells (“P2”) as described above by first culturing in MM/FGF and then switching to MM media. P2 RPE cells were cultured for 14 days and harvested by 10× TrypLE treatment for 15 min and then cryopreserved. The cells were then thawed, formulated in GS2, and underwent quality testing. Results are shown in Table 1.

In a second experiment, Mitomycin-C inactivated HDF cells were plated onto an iMatrix511 (Takara Bio)-coated well. J1 hES cells were then plated onto the iMatrix511-HDF well and cultured for 8 days in StemFit media (Ajinomoto). The media was then switched to EBDM to promote RPE generation. EBDM was changed every day for 7 days, then changed every 2-3 days.

After 47 days (approximately 7 weeks) in EBDM, the cells were treated with collagenase for six hours. The released digested material was passed through a column of strainers consisting of a 100 micron strainer resting atop a 40 micron strainer sitting on a collection tube. The clusters retained on the 40 micron strainer were recovered and dissociated into single cells by 10× TrypLE treatment for 15 min. The single cells were plated onto iMatrix511-coated wells in EBDM and EBDM was changed every 2-3 days. After 39 days (approximately 5 weeks) in EBDM after being re-plated, cells were treated with collagenase overnight. The released digested material was passed through a column of strainers consisting of a 100 micron strainer resting atop a 40 micron strainer sitting on a collection tube. The clusters retained on the 40 micron strainer were recovered and dissociated into single cells by 10× TrypLE (Thermo Fisher) treatment for 15 min. The single cells were plated as passage 0 RPE cells (“P0”) onto gelatin-coated wells in MM/FGF media. The MM/FGF media was changed every day until about >90% confluent and then changed to MM media every 2 days until harvest. P0 RPE cells were cultured for 16 days. P0 cells were harvested by 10× TrypLE treatment for 15 min and single cells were again plated as passage 1 RPE cells (“P1”) onto gelatin-coated wells in MM/FGF media. Culture method was repeated as described above for P0 RPE cells by first culturing in MM/FGF and then switching to MM media. P1 RPE cells were cultured for 14 days. P1 RPE cells were harvested and replated as passage 2 RPE cells (“P2”) as described above by first culturing in MM/FGF and then switching to MM media. P2 RPE cells were cultured for 14 days and harvested by 10× TrypLE treatment for 15 min and then cryopreserved. The cells were then thawed, formulated in GS2, and cultured on gelatin (for certain tests), and underwent quality testing. Results are shown in Table 2.

Quality testing was performed as generally described in US Pub. No. 2015/0366915, which is hereby incorporated by reference in its entirety. For example, purity (MITF/PAX6), bestrophin, and ZO1 levels were determined by immunofluorescence assay (IFA).

Phagocytosis/potency assay is performed as described in WO 2016/154357, which is hereby incorporated by reference in its entirety.

TABLE 1

| Test (days cultured on gelatin after | RPE lot |
|---|---|
| thawing and formulating in GS2) | TD1018 |
| Recovery (day 0) | 31.1% |
| Viability (day 0) | 91.4% |
| FISH (Chr12/Chr17) (day 8) | Normal |
| Karyotype (day 3) | Normal |
| Purity MITF and/or PAX6 (day 2) | 100% |
| Potency (day 4) | 88.0% |
| Bestrophin (day 28) | 60% |
| ZO1 (day 28) | 97% |

TABLE 2

| Test (days cultured on gelatin after thawing | RPE lot |
|---|---|
| and formulating in GS2) | TD2418 |
| Recovery (day 0) | 22.1% |
| Viability (day 0) | 94.1% |
| FISH (Chr12/Chr17) (day 8) | Normal |
| Karyotype (day 3) | Normal |
| Purity MITF and/or PAX6 (day 2) | 100% |
| Potency (day 4) | 94.2% |
| qPCR for hRPE mRNA (day 0): | Pass |
| BEST1, PAX6, MITF, RPE65: up-regulated | |
| by a minimum of 1 log₁₀ compared to | |
| hESC qPCR for hESC mRNA (day 0): | |
| downregulated compared to hESC (log₁₀): | |
| OCT4: ≤−2.13 | |
| SOX2: ≤−0.63 | |
| NANOG: ≤−1.95 | |
| Bestrophin (day 28) | 83% |
| ZO1 (day 28) | 100% |

### Example 3: RPE Cells Produced by the Single RPE Progenitor Cell Subculture Method and RPE Progenitor Cell Cluster Subculture Method

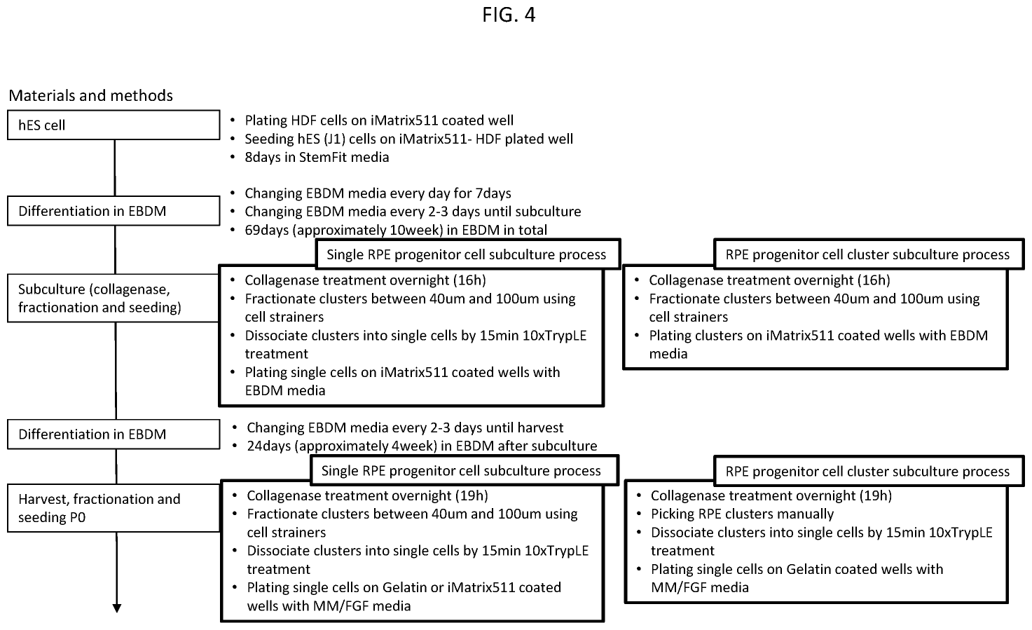

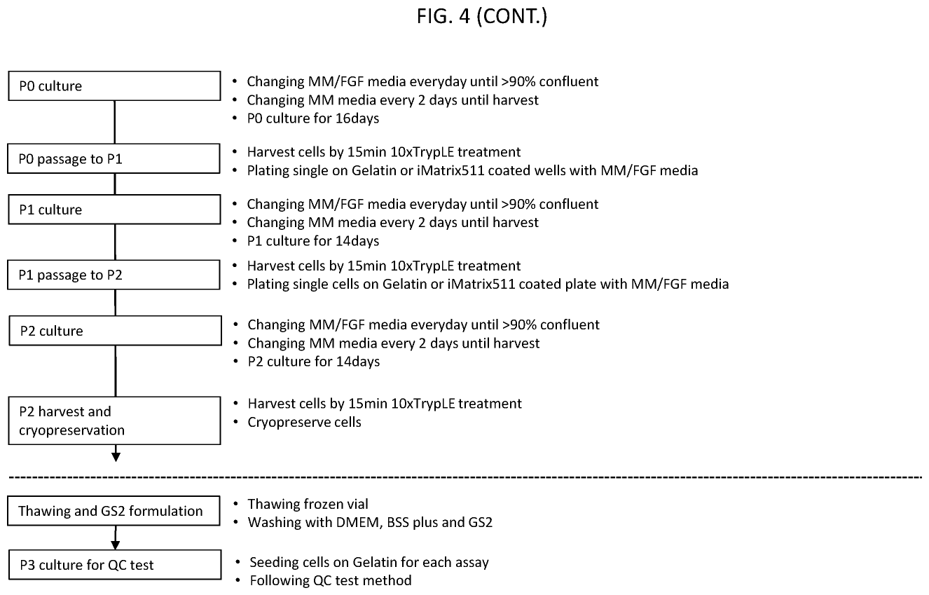

In a first experiment, RPE cells were produced by the single RPE progenitor cell subculture method and RPE progenitor cell cluster subculture method as shown in FIG. 4. Briefly, Mitomycin C-inactivated HDF cells were plated onto iMatrix511-coated wells. J1 hESCs were then plated onto the iMatrix511-HDF wells and cultured in StemFit media for 8 days. Media was then changed to EBDM to promote RPE generation. After 69 days (approximately 10 weeks) in EBDM, the cells were treated with collagenase overnight. The released digested material was passed through a column of strainers consisting of a 100 micron strainer resting atop a 40 micron strainer sitting on a collection tube. The clusters retained on the 40 micron strainer were recovered. For the single RPE progenitor cell subculture procedure, clusters were dissociated with 10× TrypLE into single cells and cultured in EBDM on iMatrix511. For the RPE progenitor cell cluster subculture procedure, clusters obtained post-collagenase and strainer fractionation were seeded intact in EBDM on iMatrix511. All seeded wells underwent EBDM medium changes every other day or every third day.

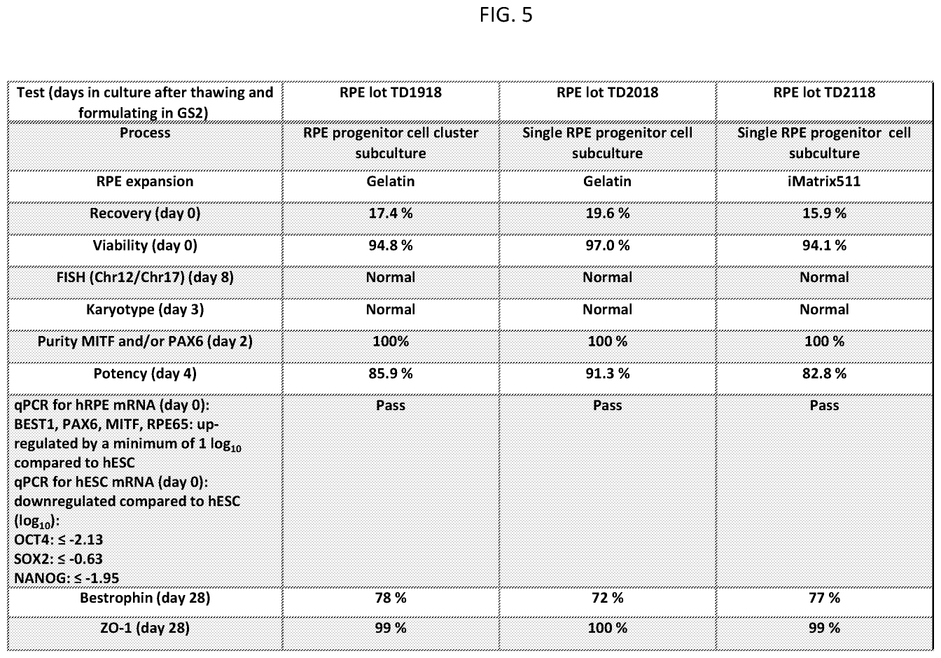

Approximately 24 days (approximately 4 weeks) in EBDM after re-plating, the wells in the single RPE progenitor cell subculture process underwent the same collagenase treatment and strainer fractionation as described above and clusters were dissociated into single RPE cells. Wells in the RPE progenitor cell cluster subculture process were treated with collagenase, strained to remove single cells and underwent negative and positive selection by inspection and manual manipulation. Isolated patches were dissociated with 10× TrypLE into single RPE cells. The single RPE cells obtained from the single RPE progenitor cell subculture and RPE progenitor cell cluster process were separately seeded as P0 RPE cells in gelatin or iMatrix511-coated wells in MM/FGF. The MM/FGF media was changed every day until about >90% confluent (about 3 days) and then changed to MM media every 2 days until harvest. The process was repeated until P2 RPE cells were obtained and cryopreserved. The cells were then thawed, formulated in GS2, cultured on gelatin (if needed), and underwent quality testing. Quality testing was performed as generally described in US Pub. No. 2015/0366915, which is hereby incorporated by reference in its entirety. For example, purity (MITF/PAX6), bestrophin, and ZO1 levels were determined by immunofluorescence assay (IFA). Phagocytosis/potency assay is performed as described in WO 2016/154357, which is hereby incorporated by reference in its entirety. Results are shown in FIG. 5.

### Example 4: Evaluation of Two Immunosuppressive Therapy Regimens as Graft Rejection Prophylaxis Following Subretinal Transplantation of RPE Cells and Proof of Concept Determination for RPE Cells as a Treatment for Atrophy Secondary to Age-Related Macular Degeneration in Patients with Moderate to Severe Visual Impairment

The human pluripotent stem cell derived retinal pigment epithelial (hPSC RPE) cells of the present disclosure can be used for subretinal transplantation as a treatment for atrophy secondary to age-related macular degeneration in patients with moderate to severe visual impairment. This study will evaluate the effectiveness, safety and tolerability of two regimens of short-term, low dose, systemic immunosuppressive therapy (IMT) as graft rejection prophylaxis after administration of hPSC RPE cells (Part 1). This study will also demonstrate the efficacy of hPSC RPE cells for atrophy secondary to age-related macular degeneration in patients with moderate to severe visual impairment (Part 2).

In Part 1 of the study, there is a sequential assessment of hPSC RPE cells with 1 of 2 immunosuppressive therapy regimens in up to 15 subjects for each regimen. The occurrence of graft failure or rejection in Part 1 determines the immunosuppressive therapy regimen used for the subsequent subjects treated in Part 2 of the study. Part 2 of the study is a proof of concept study, which includes subjects treated with the selected immunosuppressive therapy or a longer immunosuppressive therapy regimen from Part 1.

### Doses and Administration

A single dose of hPSC RPE cells and GS diluent (optional) are administered by subretinal injection to the study eye. The hPSC RPE cells dose is determined prior to treatment of the first subject in this study based on results from a separate dose escalation study, wherein a subject is treated with 50,000; 150,000; and 500,000 hPSC RPE cells.

The immunosuppressive therapy formulation comprises Prograf® 0.5 mg capsules, Prograf® 1 mg capsules, and mycophenolate mofetil (MMF) 500 mg tablets, all of which are administered orally. Prograf® is administered at an initial dose of 0.05 mg/kg per day divided into 2 daily doses and adjusted to achieve a target trough level between 3 to 5 ng/mL. The initial dose of Prograf® may need to be adjusted for subjects taking CYP3A4 inhibitors (other than protease inhibitors, direct Factor Xa inhibitors, direct thrombin inhibitors, or erythromycin) such as azole antifungals (e.g., variconazole, ketoconazole) or antibiotics (e.g., clarithromycin, chloramphenicol). MMF is administered at a dose of 1.0 g orally twice daily. There are 2 IMT regimens; during regimen 1 Prograf® and MMF are initiated 1 week prior to day of hPSC RPE cells transplant. Both the IMT drugs are continued for 6 weeks after the transplant. During regimen 2, Prograf® and MMF are taken for 1 week prior to day of transplant and are then discontinued.

hPSC RPE cells are administered to the study eye via a subretinal injection following standard 3-port pars plana vitrectomy. Subjects remain supine for at least 6 hours following transplantation. The SSC recommends the location for the cell transplant injection. The dose for hPSC RPE cells is determined by a separate dose escalation study, wherein a subject is treated with 50,000; 150,000; and 500,000 hPSC RPE cells.

Posttransplant, all subjects treated with hPSC RPE cells are assessed for safety and efficacy in the study eye at day 1, weekly from week 1 to 4 (no week 3 visit for the 1 week immunosuppressive therapy regimen), every 2 weeks from week 6 to 14, at weeks 20, 26, 52 and 78 and annually thereafter until the end of year 5. Untreated controls are assessed for efficacy in the study eye at study start reference day 0 and at weeks 4, 8, 12, 20, 26 and 52. Week 52 is the end of study (EoS) for the control group.

All adverse events (AEs) are captured from the screening visit through week 52. After that time, only AEs of special interest are captured, including all ocular and immune-mediated events.

An image reading center assesses results from fundus photography, fundus autofluorescence, spectral domain-optical coherence tomography (SD-OCT), optical coherence tomography—angiography (OCT-A), adaptive optics (AO) and fluorescein angiography (FA). A central microperimetry data collection center and central laboratory is also utilized. To the extent possible, the visual function examiners and the reading center is masked to the treatment group.

### Immunosuppressive Therapy Evaluation

Subjects first entering the study and randomized to the hPSC RPE cells treatment arm are assigned sequentially to 1 of 2 regimens of low-dose combination immunosuppressive therapy (Prograf® and mycophenolate mofetil) and infection prophylaxis as follows:

Cohort 1/immunosuppressive therapy Regimen 1: 7 weeks of immunosuppressive therapy and prophylaxis medications starting 1 week prior to day of transplantation.

Cohort 2/immunosuppressive therapy Regimen 2: 1 week of immunosuppressive therapy and prophylaxis medications starting 1 week prior to day of transplantation.

While the subject is taking the immunosuppressive therapy, the immunosuppressive therapy physician monitors the subject for safety.

Each cohort consists of up to 15 subjects treated with hPSC RPE cells. If there is 1 or no occurrence of graft failure or rejection in Cohort 1, then randomization to a treatment arm in Cohort 2 begins once Cohort 1 is fully enrolled and the last treated subject has completed the week 14 visit.

If more than 1 subject in a cohort or across the cohorts has evidence of graft failure or rejection, the immunosuppressive therapy regimen for subjects who are being treated and subjects yet to be treated is modified.

Absent attribution to another cause, graft failure or rejection consists of the following:
Evidence of unanticipated and persistent or increasing noninfectious ocular inflammation (e.g., vasculitis, retinitis, choroiditis, vitritis, pars planitis or anterior segment inflammation/uveitis).
Posttransplant appearance and then disappearance of pigmented patches on fundus photographs or hyper-reflective material above the Bruch's membrane on SD-OCT.
Within the initial 52 weeks of the study, if a gain of ≥10 letters is confirmed by a repeat measure or at the next scheduled visit, then a subsequent confirmed loss of >10 letters that cannot be attributed to another cause may be considered evidence of graft failure or rejection.
Other ocular signs or symptoms that, in the opinion of the investigator and/or the Data and Safety Monitoring Board (DSMB), that may be due to graft failure or rejection. The final determination of whether a report of “other ocular signs or symptoms” constitutes graft failure or graft rejection is made by the Sponsor, based on guidance from the DSMB.

### Efficacy

The primary analysis set will be the full analysis set, which will include all randomized, treated subjects who received the selected IMT regimen or a longer IMT regimen from the hPSC RPE groups and randomized subjects who reach day 0 from the untreated control group (from both parts of the study). The 2-sided 5% significance level will be used to assess statistical significance for all analyses.

The primary endpoint is change from baseline in the total area of atrophy at week 52. The analysis of the primary endpoint will be estimated from a mixed model repeated measures (MMRM) analysis for the change from baseline to each week (weeks 4, 8, 12, 20, 26 and 52). The model will include the following fixed effects: study group (hPSC RPE or Untreated), stratification groups of baseline area of DDAF (2 levels) and hyperAF around the area of DDAF in the study eye (2 levels), site (pooled where necessary), time (study week) and treatment-by-time interaction, as well as the covariate of baseline. Parameters will be estimated using restricted maximum-likelihood and degrees of freedom will be estimated using the Kenward-Roger approximation. The unstructured variance-covariance structure will be used to estimate the within-subject errors in the model. If the fit of the unstructured covariance structure fails to converge, other variance-covariance structure will be used until convergence. Missing data will not be imputed in this analysis.

Least squares means (with standard errors) for both study groups and study group difference of hPSC RPE versus untreated control (also with 95% confidence interval) will be shown for weeks 4, 8, 12, 20, 26 and 52.

The analysis for the secondary endpoint “subject visual function response, defined as a confirmed ≥15 letter improvement (within the visit window) in study eye” (change from baseline to week 52) will use the chi-square test for the study group comparison. If there are fewer than 5 subjects in any cell of the 2×2 table, then Fisher's Exact Test will be used instead. The proportion of subjects with ≥15 letter improvement in study eye will be shown for study groups and study group difference (with 95% confidence interval). In addition to the observed data analysis, subjects with missing values will be assessed using nonresponse for missing data.

The analysis for the secondary endpoints “change from baseline in area of atrophy in the index quadrant,” “change from baseline in mean microperimetry sensitivity of perilesional test points at week 52,” “change from baseline in log contrast sensitivity at week 52” and “change from baseline in BCVA at week 52” will be analyzed using the same MMRM model as described above for the primary endpoint. The included time points for area of atrophy will be weeks 4, 8, 12, 20, 26 and 52, for BCVA will be weeks 4, 8, 12, 20, 26 and 52 (time points common to both RPE cells and untreated groups), for microperimetry sensitivity will be weeks 4, 12, 20, 26 and 52, and for contrast sensitivity will be weeks 4, 12, 26 and 52.

The analysis of the “change from baseline” in the summary score representing all items of the Impact of Vision Impairment questionnaire (IVI) at week 52 will use an analysis of covariance (ANCOVA) model, which will include terms for study group (ASP7317 or Untreated), stratification groups of baseline area of DDAF (2 levels) and hyperAF around the area of DDAF in the study eye (2 levels) and site (pooled where necessary).

The primary and secondary endpoints will also be analyzed separately for the severe (baseline BCVA 20/320 to <20/200) and moderate (baseline BCVA 20/200 to 20/80) visual impairment groups (subject to sufficient numbers of subjects in each subgroup analysis).

The week 52/ET time point will be analyzed for all endpoints described above, using ANCOVA as described above except for “subject response, defined as a confirmed ≥15 letter improvement in study eye,” which will use the chi-square test as described above.

### Example 5: Comparison of RPE Cell Production from the Conventional Selective Picking Method without Subculture, the RPE Progenitor Cell Cluster Subculture Method with Selective Picking, and the Single RPE Progenitor Cell Subculture Method without Selective Picking

A comparison of RPE cell production was made between the 1) conventional RPE cell production method involving labor intensive selective picking without subculture, 2) RPE progenitor cell cluster subculture method with selective picking described herein, and 3) the single RPE progenitor cell subculture method without selective picking described herein. The conventional RPE cell production method was performed as generally described in WO 2005/070011 via the adherent hES monolayer method. Briefly, J1 hES cells were differentiated on HDF in EBDM for 90-100 days until pigmented patches with polygonal, cobblestone morphology and brown pigment in the cytoplasm were formed. These pigmented polygonal cells were digested and the pigmented islands were selectively picked manually. The picked pigmented clusters were dissociated into single cells, counted, and seeded as P0 RPE cells. RPE cells obtained from the RPE progenitor cell cluster subculture method with selective picking and single RPE progenitor cell subculture method without selective picking were similarly counted before seeding as P0 RPE cells.

Table 3 shows the RPE cells produced from methods involving selective picking: the conventional selective picking method without subculture and the RPE progenitor cell cluster subculture method with selective picking of the present invention. Table 3 shows that the RPE progenitor cell cluster subculture method with selective picking can produce a larger number of cells per lot compared to the conventional method, but more significantly, that the RPE progenitor cell cluster subculture method with selective picking produced a greater average number of cells per hour of manual labor required to selectively pick RPE cells compared to the conventional method. Additionally, because the conventional method did not involve the subculture step where RPE progentitors are concentrated, selective picking from the less pure populations of the conventional method resulted in less cells obtained, greater variability in morphology, and longer labor time to selectively pick RPEs.

Table 4 shows the RPE cells produced from the single RPE progenitor cell subculture method that does not involve manual, selective picking of RPE cells. The single RPE progenitor cell subculture method produced significantly more RPE cells than the conventional method or the RPE progenitor cell cluster subculture method with selective picking. Moreover, the total number of cells obtained per hour taken to isolate P0 RPE cells was also significantly higher.

The methods of the invention provide significant improvements over the conventional method that requires manual, selective picking of RPE cells from a less pure population. Manual picking is physically and mentally demanding and requires several hours of continuous work with extreme precision and undivided attention for several days to make one decently sized lot. Training of new operators on the conventional method is also challenging because it requires both precise mechanical operation under the microscope and experience with cell morphologies since a small number of contaminating cells, if mistakenly accepted, can overgrow RPE resulting in lot failure. Each picked cluster needs to be evaluated by the operator for morphology before it is accepted or rejected. Some clusters may have other than ideal RPE morphology, and the operator needs to make a subjective decision whether to accept or reject the cluster. Once each cluster is evaluated, it needs to be quickly moved. This procedure is repeated 2-3 times to eliminate single cells and ensure the quality of picked clusters. Slow speed by the operator could result in very low yields and decision-making errors could result in low purity and lot failure. Thus, a skilled operator needs to have experience with aseptic procedures, proficiency with micro-manipulations under the microscope in the sterile environment, experience enabling relatively fast moving of selected and rejected clusters, experience with cell morphology enabling fast decision making about each cluster evaluated. The methods of the present invention allow the use of standard cell culture methods which can be used by personnel with minimal cell culture experience, and the cell yields are significantly greater.

TABLE 3

| | | | Average cell number | | Purity by |
|---|---|---|---|---|---|
| | | Total | per hour of selective | | Pax6+ or |
| | | cells | picking of P0 | | MITF+ of |
| Method | Lot # | per lot | RPE cells per lot | | P0 RPE |
| Conventional | 1 | 9,626 | 3,209 | | N/A |
| selective | 2, 3, | 282,241 | 6,135 (ave from 3 lots) | | N/A |
| picking without | 4 | (ave from | | | |
| subculture | | 3 lots) | | | |
| RPE Progenitor | 5 | 51,775 | 26,551 | 39,770 | N/A |
| Cell Cluster | 6 | 107,000 | 35,666 | | 99% |
| Subculture | 7 | 333,000 | 111,000 | | 100% |
| method with | 8 | 107,000 | 23,000 | | 98% |
| selective | 9 | 72,000 | 14,400 | | 100% |
| picking | 10 | 142,000 | 28,000 | | N/A |

TABLE 4

| | | | Ave cell | |
|---|---|---|---|---|
| | | | number per | Average |
| | | Ave. | hour taken | hours to |
| | Lot* | total cells | to isolate P0 | isolate P0 |
| Method | # | per lot | RPE cells | RPE cells |
| Single RPE | 11, | 48,222,500 | 8,037,083 | 6 hrs |
| Progenitor Cell | 12, | | | |
| Subculture method | 13, | | | |
| w/o selective | 14 | | | |
| picking | | | | |

*No IFA was performed or P0 RPE cells. However, all four lots passed QC testing with >95% purity at P1.

1. A method for producing a population of retinal epithelium (RPE) cells, the method comprising:
(i) obtaining cell clusters of PAX6+/MITF+ RPE progenitor cells and dissociating the cell clusters into single cells;
(ii) culturing the single cells in a differentiation medium such that the cells differentiate to RPE cells; and
(iii) harvesting the RPE cells produced in step (ii);
thereby producing a population of RPE cells.

2. A method for producing a population of retinal epithelium (RPE) cells, the method comprising:
(i) obtaining cell clusters of PAX6+/MITF+ RPE progenitor cells,
(ii) culturing the cell clusters in a differentiation medium such that the cells differentiate to RPE cells; and
(iii) harvesting the RPE cells produced in step (ii);
thereby producing a population of RPE cells.

3. The method of claim 1, further comprising harvesting the RPE cells produced in step (ii) by:
(a) dissociating the RPE cells, fractionating the RPE cells, collecting RPE cell clusters, dissociating the RPE cell clusters into single RPE cells, and culturing the single RPE cells; or
(b) dissociating the RPE cells, collecting RPE cell clusters, and selectively picking RPE cell clusters.

4-5. (canceled)

6. The method of claim 1, wherein the PAX6+/MITF+ RPE progenitor cells are obtained from a population of pluripotent stem cells.

7. The method of claim 6, wherein the pluripotent stem cells are human embryonic stem cells or human induced pluripotent stem cells.

8. The method of claim 1, further comprising expanding the RPE cells.

9. The method of claim 8, wherein the RPE cells are expanded by culturing the cells in maintenance media supplemented with FGF.

10-12. (canceled)

13. The method of claim 1,
wherein the RPE cells are
(i) passaged up to two times; and/or
(ii) cryopreserved following harvesting.

14-18. (canceled)

19. The method of claim 1, wherein:
(i) the cells are cultured on feeder cells or under feeder-free conditions;
(ii) the cells are cultured in an adherent culture or in a non-adherent culture; and/or
(iii) any one of the dissociation steps is carried out by treating the cells with a dissociation reagent.

20-23. (canceled)

24. The method of claim 1, wherein
(i) the differentiation medium comprises one or more differentiation agents selected from the group nicotinamide, a transforming factor-β (TGFβ) superfamily (e.g., activin A, activin B, and activin AB), nodal, anti-mullerian hormone (AMH), bone morphogenetic proteins (BMP) (e.g., BMP2, BMP3, BMP4, BMP5, BMP6, and BMP7, growth and differentiation factors (GDF)), WNT pathway inhibitor (e.g., CKI-7, DKK1), a TGF pathway inhibitor (e.g., LDN193189, Noggin), a BMP pathway inhibitor (e.g., SB431542), a sonic hedgehog signal inhibitor, a bFGF inhibitor, nicotinamide and a MEK inhibitor (e.g., PD0325901); and/or
(ii) the differentiation medium further comprises heparin and/or a ROCK inhibitor.

25-26. (canceled)

27. The method of claim 1, wherein the cell clusters of PAX6+/MITF+RPE progenitor cells are between about 40 μm and about 200 μm in size; or between about 40 μm and about 100 μm in size.

28. (canceled)

29. The method of claim 1, wherein in step (ii), the cells are cultured on an extracellular matrix selected from the group consisting of laminin or a fragment thereof, fibronectin, vitronectin, Matrigel, CellStart, collagen, and gelatin.

30. The method of claim 29, wherein the extracellular matrix is laminin or a fragment thereof.

31. The method of claim 30, wherein the laminin is selected from laminin-521 and laminin-511.

32. (canceled)

33. The method of claim 1, wherein the duration of culturing in step (ii) is: about 1 week to about 8 weeks, at least about 3 weeks, or about 6 weeks.

34-35. (canceled)

36. The method of claim 3, wherein the RPE cell clusters are between about 40 μm and 200 μm in size, or about 40 μm and 100 μm in size.

37. (canceled)

38. The method of claim 3, wherein the single RPE cells are cultured in a medium that supports RPE growth or differentiation.

39. The method of claim 38, wherein the single RPE cells are cultured on an extracellular matrix selected from the group laminin or a fragment thereof, fibronectin, vitronectin, Matrigel, CellStart, collagen, and gelatin.

40-41. (canceled)

42. The method of claim 1, wherein the population of RPE cells are at least 75% pure, at least 80% pure, at least 90% pure, at least 95% pure, at least 96% pure, at least 97% pure, at least 98% pure, or at least 99% pure.

43. The method of claim 1, wherein the RPE cells are human RPE cells.

44. A method for producing a population of retinal epithelium (RPE) cells, the method comprising:
(i) culturing a population of pluripotent stem cells in a first differentiation medium, such that the cells differentiate into RPE progenitor cells;
(ii) dissociating the RPE progenitor cells, fractionating the cells to collect cell clusters, dissociating the cell clusters into single cells, and subculturing the single cells in a second differentiation medium such that the cells differentiate to RPE cells; and
(iii) harvesting the RPE cells produced in step (ii)
thereby producing a population of RPE cells.

45. A method for producing a population of retinal epithelium (RPE) cells, the method comprising:
(i) culturing a population of pluripotent stem cells in a first differentiation medium, such that the cells differentiate into RPE progenitor cells;
(ii) dissociating the RPE progenitor cells, fractionating the cells to collect cell clusters, and subculturing the collected cell clusters in a second differentiation medium such that the cells differentiate to RPE cells; and
(iii) harvesting the RPE cells produced in step (ii)
thereby producing a population of RPE cells.

46-48. (canceled)

49. The method of claim 44, wherein the RPE progenitor cells are positive for PAX6/MITF.

50. The method of claim 44, further comprising expanding the RPE cells.

51. The method of claim 50, wherein the RPE cells are expanded by culturing the cells in maintenance media supplemented with FGF.

52-62. (canceled)

63. The method of claim 44, wherein prior to step (i), the pluripotent stem cells are cultured:
(a) on feeder cells in a medium that supports pluripotency; or
(b) feeder-free in a medium that supports pluripotency.

64-65. (canceled)

66. The method of claim 44, wherein step (i), (ii), and/or (iii) is performed in a non-adherent culture, or in an adherent culture.

67. (canceled)

68. The method of claim 44, wherein the first and second differentiation medium are the same, or are different.

69-74. (canceled)

75. The method of claim 44, wherein the duration of culturing in step (i) is: about 1 weeks to about 12 weeks, at least about 3 weeks, or about 6 to about 10 weeks.

76-79. (canceled)

80. The method of claim 44, wherein in step (ii), the cells are subcultured on an extracellular matrix selected from the group laminin, fibronectin, vitronectin, Matrigel, CellStart, collagen, and gelatin.

81. The method of claim 80, wherein the extracellular matrix comprises laminin or a fragment thereof.

82. The method of claim 81, wherein the laminin or fragment there of is selected from laminin-521 and laminin-511.

83-93. (canceled)

94. The method of claim 1, wherein the RPE cells express one or more of markers selected from the group consisting of RPE65, CRALBP, PEDF, Bestrophin, MITF, OTX2, PAX2, PAX6, premelanosome protein (PMEL or gp-100), tyrosinase, and ZO1.

95-97. (canceled)

98. The method of claim 1, wherein the RPE cells lack substantial expression of one or more stem cell markers selected from the group consisting of OCT4, NANOG, Rex-1, alkaline phosphatase, SOX2, TDGF-1, DPPA-2, DPPA-4, stage specific embryonic antigen (SSEA)-3 and SSEA-4, tumor rejection antigen (TRA)-1-60 and TRA-1-80.

99-100. (canceled)

101. A composition comprising a population of RPE cells produced by the method of claim 1.

102. A pharmaceutical composition comprising a population of RPE cells produced by the method of claim 1 and a pharmaceutically acceptable carrier.

103. A method of treating a patient with or at risk of a retinal disease, the method comprising administering to the patient an effective amount of the composition of claim 101.

104. (canceled)
